(19) United States
(12) Patent Application Publication
(10) Pub. No.: US 2025/0205400 A1
(43) Pub. Date: Jun. 26, 2025

(54) ZWITTERIONIC POLYMER COATING WITH SURFACE GRAFT CROSSLINKING, PREPARATION METHOD AND APPLICATION THEREOF

(71) Applicant: TIANJIN UNIVERSITY, Tianjin (CN)

(72) Inventors: Shuangyang LI, Tianjin (CN); Anjie DONG, Tianjin (CN); Jianhua ZHANG, Tianjin (CN); Yiqun ZHANG, Tianjin (CN); Lingyuan LIU, Tianjin (CN)

(21) Appl. No.: 19/000,889

(22) Filed: Dec. 24, 2024

Related U.S. Application Data
(63) Continuation of application No. PCT/CN2023/090614, filed on Apr. 25, 2023.

(30) Foreign Application Priority Data
May 12, 2022 (CN) 202210519886.5
Jul. 19, 2022 (CN) 202210852497.4

(51) Int. Cl. A61L 33/00; A61L 27/34; A61L 27/50; A61L 31/10; A61L 33/06; C08J 3/24; C08J 7/16; C09D 4/00; C09D 5/16; C09D 133/14

(52) U.S. Cl. CPC A61L 33/0088; A61L 27/34; A61L 27/507; A61L 31/10; A61L 33/068; C08J 3/245; C08J 7/16; C09D 4/00; C09D 5/1668; C09D 133/14; A61L 2400/18; A61L 2420/02; A61L 2430/34; C08J 2327/06; C08J 2367/02; C08J 2375/04; C08J 2377/00; C08J 2383/04; C08J 2433/14

(57) ABSTRACT

A zwitterionic polymer coating with surface grafting crosslinking, its preparation method and application are disclosed, which relate to surface modification of biomedical materials, pipelines and other medical equipment, such as extracorporeal blood circulation devices, artificial blood vessels, catheters, endoscopes and other medical equipment, to provide functions such as anticoagulation and anti-biological adhesion. The disclosure constructs the zwitterionic polymer coating with simple process and excellent performance by simulating the structure of the blood vessel intima, having the surface micro-nano structure similar to the blood vessel intima, extremely low friction coefficient, soft elasticity and super hydrophilicity, realizing zero activation and zero adhesion of blood platelets, further providing the blood circulation effect of “blood undisturbed”, which is beneficial to avoiding the problems of thrombus of artificial blood vessels in vivo, various complications caused by clinical blood extracorporeal circulation and the like, and has great clinical application value.

### TECHNICAL FIELD

The disclosure relates to a zwitterionic polymer coating with surface graft crosslinking, its practical application, mainly relating to surface modification of biomedical materials, pipelines and other medical equipment, such as extracorporeal blood circulation devices, artificial blood vessels, catheters, endoscopes and other medical equipment, providing good “blood undisturbed” blood circulation effects such as anticoagulation, anti-bioadhesion, and inhibition of platelet activation.

### BACKGROUND ART

Extracorporeal blood circulation is an important means commonly used in hemodialysis, clinical cardiac surgery and the like to perform blood purification or temporarily replace cardiopulmonary function. One of the serious complications faced by the clinical use of these extracorporeal circulation devices is thrombosis. For example, the extracorporeal membrane oxygenation machine (ECMO), a ventilator known as a “life-saving machine” especially when the global new coronavirus raging, is mainly used for long-time cardiopulmonary replacement therapy in patients with severe cardiopulmonary failure. However, during the extracorporeal circulation of blood, contact between blood and non-endothelial surfaces can lead to platelet activation, which in turn leads to thrombosis and blood destruction, so that blood anticoagulation management is generally required, heparin is most commonly used, but long-time or excessive use of heparin induces thrombocytopenia (HIT), and there is a risk of bleeding and other complications. The current extracorporeal blood circulation materials mainly include polyvinyl chloride, polycarbonate, polyurethane and polypropylene, various biological reactions can be induced between the extracorporeal blood circulation materials and blood, such as protein adhesion, platelet adhesion, coagulation and hemolysis, and a series of blood component activation reactions are generated, a large number of inflammatory factors are released, and poor clinical prognosis is caused. Therefore, to meet the clinical demands of long-time extracorporeal blood circulation, anticoagulation, anti-bioadhesion coating modification of circulatory systems, arteriovenous cannulas, artificial blood vessels, and the like is a necessary approach.

The anticoagulation coating materials developed at present mainly include two materials, namely heparin coating materials and non-heparin coating materials. Heparin coating has the characteristics of inhibiting blood component activation, reducing inflammatory factor release and the like, and is initially applied to improve coagulation response caused by extracorporeal circulation, but the heparin coating is used with bleeding risk, which can cause thrombocytopenia and a certain allergic risk. Thus, non-heparin anticoagulant coatings are becoming a new direction of development. The high hydrophilic surface of the polymer coating not only endows the polymer coating with good antifouling capability, but also can provide a soft biocompatible interface for biological tissues, thus being an ideal antifouling coating material. There are currently two methods for polymer coating. One is to introduce reactive active groups on the surface of a substrate and then graft water-soluble macromolecules on the surface to form a “macromolecular brush”-type hydrogel thin layer, the thickness of which is between hundreds of nanometers and a few micrometers, however, the coating prepared by this method is too thin to withstand mechanical damage such as shear and friction, resulting in coating detachment. The other method is to form a polymer coating with a cross-linked network structure with the thickness of more than 50 μm by a molding or dipping coating mode on the surface, but the coating formed by this method has poor mechanical compatibility between the coating and the pipeline body, due to the difficulty to control the microstructure of the surface of the coating. At present, when the polymer coating prepared by above method is contacted with blood for a long time, the activation and adhesion of platelets can only be reduced, but cannot be avoided completely, obversing thrombus can still occur on the surface of the hydrogel.

Among the numerous hydrogel materials, zwitterionic hydrogels exhibit more excellent blood compatibility due to their excellent hydration ability, and are effective against the adhesion of proteins, bacteria, cells, and platelets to their surfaces, thereby inhibiting thrombosis. Zwitterionic polymers are widely used in surface modification coatings for resisting bioadhesion, various methods used for preparing the zwitterionic polymer coating in the meanwhile. The gel strength is weakened due to the tendency of zwitterionic polymer coating to absorb water and swell easily, which can particularly-cause larger stress in a hydrogel thin layer to damage the bonding force between the coating and substrate, as a result, the zwitterionic polymer coating being easily peeled off from the surface of the substrate. Crosslinking, inhibiting swelling of zwitterionic hydrogels, results in gels that are brittle and have poor bonding with substrates. Therefore, zwitterionic polymer coatings have difficulty in being applied into practical use.

The intima of natural blood vessels in contact with blood, consisting of endothelial cell layers and surrounding longitudinal elastic fibers and connective tissue, is the best blood compatible surface. The surface of intima is distributed with a single endothelial cell layer along the long axis direction of the blood vessel, with a submicron-scale groove and a nanometer-scale package-shaped protruding structure on the surface of the groove. The surface of endothelial cells is covered with a high-hydrophilicity gel layer of glycoprotein composite structure, which has soft elasticity (surface Young's modulus: 1-100 kPa) and super lubricity (friction coefficient: 0.04-0.15), providing good dynamic environment for blood flow. In addition, research shows that the nanometer-micrometer topological structure on the surface of the simulated blood vessel intima can effectively inhibit the activation and adhesion of platelets, which are the determining factors of the anticoagulation performance of the surface of the material. Therefore, the disclosure simulates the structure of the blood vessel intima, constructing a zwitterionic polymer coating with simple process and excellent performance, which has the surface nanometer-micrometer structure similar to the blood vessel intima, demonstrating extremely low friction coefficient, soft elasticity and super hydrophilicity, so that zero activation and zero adhesion of platelets are realized (observed), and the blood circulation effect of “blood undisturbed” is provided furthermore.

### SUMMARY

In view of the above, the present disclosure aims to provide a coating suitable for anticoagulation and anti-bioadhesion of different material surfaces and its preparation technology, in particular, a zwitterionic polymer coating with surface graft crosslinking, its preparation method and application.

The zwitterionic polymer coating with surface graft crosslinking prepared by the disclosure has the surface topological structure of micro-nano grooves and nano-micro holes like the blood vessel intima, demonstrating super hydrophilicity, lower surface Young's modulus and low friction coefficient, showing the property of “blood undisturbed” in the long-time contact process with blood, which has the anticoagulation effect of zero platelet activation and zero thrombosis, along with firm adhesion, good mechanical strength, stability of anti-scouring, wear resistance and the like bending resistance. In addition, the coating has the characteristics of convenient for application and conversion with simple and convenient process, wide application, low cost and the like.

In order to achieve the above purpose, the present disclosure adopts the following technical scheme:
A zwitterionic polymer coating with surface graft crosslinking is provided, which is a coating formed in the surface of a substrate by simultaneously initiating a graft crosslinking polymerization of a zwitterionic monomer and a water-soluble crosslinking agent both in the surface of a polymer substrate and an aqueous solution which has been activated in advance by a surface initiator;
Wherein the thickness of the polymer coating is 25-100 μm;
The friction coefficient of the polymer coating in an aqueous medium is less than 0.005, and the surface Young's modulus is 10-60 kPa.

The aqueous solution contains zwitterions, water-soluble crosslinking agents and water-soluble initiators. The concentration of the zwitterionic monomer is 10 wt % to 60 wt %, and the water-soluble initiators account for 0.5 wt % to 20 wt % of the mass of the zwitterionic monomers; and the water-soluble crosslinking agents functioning by chemical bonding accounting for 3 wt % to 12 wt %, and the physical crosslinking agent accounting for 0 wt % to 40 wt % both of the mass of the zwitterionic monomers.

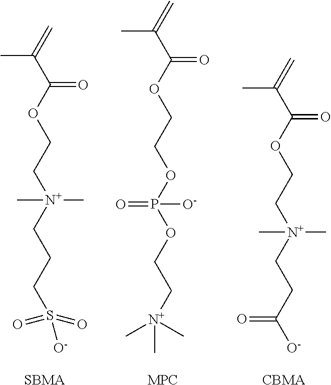

Wherein the zwitterionic monomer is at least one of methacryloyl ethyl sulfobetaine (SBMA), 2-methacryloyl oxyethyl phosphorylcholine (MPC) and carboxylic acid betaine methacrylate (CBMA).

The chemical crosslinking agent at least contains one of N, N-methylenebisacrylamide (MBA), N, N-bis (acryloyl) cystamine (MSBA), ethylene glycol dimethacrylate (EBA), and carboxylate betaine dimethacrylate (CBBA).

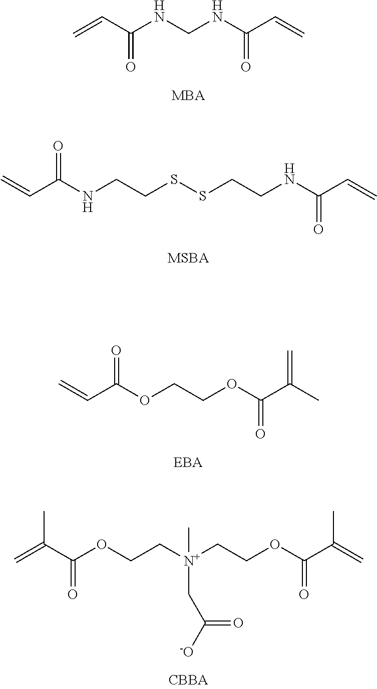

The aforementioned surface initiator is preferably hydrophobic, as it favors the graft cross-linking polymerization reaction at the interface between the surface and the aqueous phase.

Appropriate crosslinking can enhance the coating thickness and stability and impart excellent mechanical strength including resistance to erosion, abrasion, and bending, and the coating possesses a surface topology characterized by crosslinked micrometer-scale grooves and nano- to micrometer-scale pores, combining the super hydrophilicity, extremely low friction coefficient (<0.005) and the surface Young's modulus (10-60 kPa) with soft elastic characteristics originating from zwitterions, and providing excellent protein adhesion resistance, zero platelet adhesion and long-acting anticoagulation performance.

Further, the physical crosslinking is selected from the group consisting of N-acryloylglycine amide (NAGA).

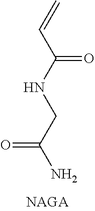

NAGA, as a monomer with strong hydrogen bond forming capability, is often used as a physical crosslinking agent to be introduced into a hydrogel network, and the formed hydrogen-bonded physical crosslinking network exhibits reversibility, so that the strength and toughness of the hydrogel are greatly improved. The NAGA unit is introduced into the coating of the disclosure, as a hydrogen bond physical crosslinking dynamic network added on the basis of chemical crosslinking, significantly enhancing the mechanical strength and the surface grafting stability of the coating, which results in the coating has better wear resistance, water scouring resistance and bending resistance, making it suitable for the application requirements of artificial blood vessels, extracorporeal blood circulation and other longer-term anticoagulant coatings.

The polymer coating is a coating formed on the surface with a polymer as a substrate. Surface initiator penetrates into the polymer surface and activates it, therefore, the grafted crosslinked zwitterionic polymer coating is more stable.

The substrate materials include, but are not limited to, polyvinyl chloride (PVC), polyurethane (PU), polydimethylsiloxane (PDMS), polycarbonate (PC), polyethylene terephthalate (PET), various rubbers, and other polymer materials.

The surface of the polymer substrate and the aqueous solution are simultaneously initiated with graft crosslinking polymerization of the zwitterionic monomer and the water-soluble crosslinking agent to form a coating, wherein the concentration of the zwitterionic monomer in the aqueous solution is preferably 15 wt %-40 wt %, the chemical crosslinking agent accounting for 5 wt %-10 wt % of the mass of the zwitterionic monomer, the physical crosslinking agent accounting for 10 wt %-40 wt % of the mass of the zwitterionic monomer, and the water-soluble initiator accounting for 1 wt %-15 wt % of the mass of the zwitterionic monomer. The prepared coating has a crosslinked micro-nano groove structure on the surface, and has better stability and anti-adhesion performance. Because the crosslinked structure is favorable for improving the strength of the coating, the mechanical stability of the coating is poor under the condition of too low crosslinking agent dosage, while using too high an amount increases the brittleness of the coating and reduces its hydrophilicity, leading to an increase in protein and platelet adhesion. On the other hand, the graft polymerization efficiency is lowered when the monomer concentration is too low or the amount of initiator is too small, but too much monomer and initiator cause it difficult to control the reaction and the coating structure.

The disclosure also claims the protection of a method for preparing the zwitterionic polymer coating with surface graft crosslinking, which is characterized in that a surface initiator is firstly swelled into a modified surface, and then graft crosslinking polymerization of a zwitterionic monomer and a water-soluble crosslinking agent is jointly initiated in the surface and an aqueous solution to obtain the surface coating; the preparation method includes the following specific steps:
(1) soaking the substrate in a solution of a surface initiator to diffuse the surface initiator into a surface layer of the substrate, activating the surface of the substrate, flushing the surface of the substrate with deionized water or a solvent, and drying;
(2) immersing the surface of the substrate activated by the surface initiator into a zwitterionic polymer precursor solution, performing a graft crosslinking polymerization by photo or thermal initiation, and then flushing the adsorbate on the surface by deionized water to form a zwitterionic polymer coating with a graft crosslinking structure on the surface of the substrate;
Wherein the precursor solution is an aqueous solution composed of a zwitterionic monomer, a water-soluble crosslinking agent and a water-soluble initiator, the concentration of the zwitterionic monomer is 10 wt %-60 wt %, the chemical crosslinking agent accounting for 3 wt %-12 wt % of the mass of the zwitterionic monomer, the physical crosslinking agent accounting for 0 wt %-40 wt % of the mass of the zwitterionic monomer, and the water-soluble initiator accounting for 0.5 wt %-20 wt % of the mass of the zwitterionic monomer.

Also, the surface initiator is a photo initiator or thermal initiator, preferably hydrophobic benzophenone, 4-methyl benzophenone, isopropyl thioxanthone, benzoyl peroxide or azodiisobutyronitrile; the water-soluble initiator is a photo initiator or a thermal initiator selected from Irgacure-2959, α-ketoglutaric acid, ammonium persulfate or potassium persulfate.

The method of a graft crosslinking polymerization by photo initiation includes the following specific steps:
(1) immersing the modified surface of the substrate in a surface photo initiator solution, performing activation treatment on the surface of the substrate, then flushing the surface with deionized water or a solvent, and drying; wherein the surface photo initiator is selected from hydrophobic benzophenone, 4-methyl benzophenone or isopropyl thioxanthone
(2) immersing the surface of the substrate activated by the surface initiator in the step (1) into a zwitterionic polymer precursor solution, initiating surface graft crosslinking polymerization by ultraviolet light, and then flushing the surface of the substrate by deionized water to form a zwitterionic polymer coating with a graft crosslinking structure on the surface of the substrate;
In the zwitterionic polymer precursor solution, the concentration of the zwitterionic monomer is 10 wt %-60 wt %, the chemical crosslinking agent accounting for 3 wt %-12 wt % of the mass of the zwitterionic monomer, the physical crosslinking agent accounting for 0 wt %-40 wt % of the mass of the zwitterionic monomer, and the water-soluble photon initiator accounting for 0.5 wt %-20 wt % of the mass of the zwitterionic monomer, which is selected from Irgacure-2959 or α-ketoglutaric acid.

Also, the surface photo initiator is preferably benzophenone, and the water-soluble photo initiator is preferably Irgacure-2959. Benzophenone and Irgacure-2959 are photo initiators with better biological safety and are commonly used for preparing biological materials.

The photo-initiated preparation of the coating is preferably carried out on a polymer substrate material, including polyvinyl chloride, polyurethane, polyester, polyamide or rubber; the concentration of the zwitterionic monomer in the aqueous solution is 15-40 wt %, the water-soluble initiator accounting for 1-15 wt % of the mass of the zwitterionic monomer, the chemical crosslinking agent accounting for 5-10 wt % of the mass of the zwitterionic monomer, and the physical crosslinking agent accounting for 10-40 wt % of the mass of the zwitterionic monomer; the physical crosslinking agent is N-acryloylglycine amide, and the chemical crosslinking agent is at least one of N, N-methylenebisacrylamide, N, N-bis (acryloyl) cystamine, ethylene glycol dimethacrylate, and carboxylate betaine dimethacrylate.

The preparation method of the coating can be used for simultaneously modifying the inner surface and the outer surface of the pipeline, or alternatively, applying the coating only to the inner or outer surface, and only needs to carry out the activation and graft crosslinking process on the surface to be modified, for example, the modification of zwitterionic polymer coating on the inner surface of the pipeline which is prepared by the following steps:
(1) flushing a pipeline to be modified with isopropanol and deionized water, completely drying with nitrogen flow, sealing one end of the pipeline, pouring a surface photo initiator solution into the pipeline, performing activation treatment on the surface of a substrate, recycling the photo initiator solution in the pipeline, cleaning the pipeline with water, and drying;
(2) sealing one end of the pipeline with the surface activated, injecting a zwitterionic polymer precursor solution into the pipeline from the other end, uniformly irradiating the pipeline by ultraviolet light to initiate graft crosslinking polymerization, and then flushing unreacted substances by deionized water to obtain the zwitterionic polymer coating with surface graft crosslinking.

Furthermore, the zwitterionic monomer is preferably methacryloyl ethyl sulfobetaine, with N, N-methylenebisacrylamide as the chemical crosslinking agent, and the two raw materials are easy to obtain and low in cost, so that those application is relatively wide.

In the preparation method of the present disclosure, before being activated, the substrate is preferably flushed with a solvent such as isopropanol and water and dried. The cleaned substrate and the activated substrate can be dried in the air, but preferably under a nitrogen flow to avoid contamination.

In addition, the disclosure also claims the application of the zwitterionic polymer coating with surface graft crosslinking, which is characterized by being used for modifying the inner or outer surfaces of materials, articles and devices, endowing the surfaces with anti-bioadhesion and anticoagulation functions, and being particularly used for the surface modification application of biomedical materials, pipelines, artificial blood vessels and various medical equipment.

It should be noted that the prepared zwitterionic polymer coating with surface graft crosslinking can modify the inner and outer surfaces of pipelines and the inner and outer surfaces of various complex irregular devices simultaneously by adopting the technology of the disclosure. In addition, as long as the surface of the substance can be modified with or activated by the initiator, the technology can be adopted to form the zwitterionic polymer coating with the graft crosslinked structure on the surface of the substance, thereby playing roles in anti-bioadhesion and anticoagulation.

Compared with the prior art, the disclosure provides a zwitterionic polymer coating with surface graft crosslinking, and its preparation method and application, and has the following beneficial effects:
1) The disclosure creatively selects the zwitterionic polymer coating with surface grafting crosslinking structure, which well combines the good blood compatibility (anti-bioadhesion and extremely low interaction with blood components) of the zwitterionic polymer with the surface micro-nano structure, super lubrication and flexible elasticity characteristics of the blood vessel-like intima, realizing the extremely low biological adhesion effect of the coating, with zero activation and zero adhesion on blood platelets, efficiently resisting blood coagulation. It does not influence the blood components and the interaction of the blood components, as well as the change the “blood undisturbed” performance of the morphology of blood cells, which is beneficial to avoiding various complications caused by clinical blood extracorporeal circulation and artificial blood vessel blood internal circulation, and has great clinical application value.
2) The preparation technology of the zwitterionic polymer coating of the disclosure is simple, as the raw materials used are convenient to prepare and low in cost, and especially the coating preparation conditions of photoinitiated polymerization is mild, which does not influence the structure, the size, the shape and the body performance of the product, and is suitable for coating modification of the surface of a substrate with irregular shape; in addition, the technology is suitable for surface modification of various materials, and provides a new method for surface functionalization of different materials and different morphologies.
3) The graft crosslinking polymerization method jointly initiated by the surface and the solution can well control the thickness and the surface micro-nano structure of the coating, and the proper chemical crosslinking along with hydrogen bond physical crosslinking structure in the coating also provides stronger mechanical stability and is favorable for maintaining the surface nano-micro topological structure, so that the good combination of functions and strength is realized, and the problems of low strength, unstable combination with the surface of the substrate, poor stability and the like of the zwitterionic polymer coating are solved.

### BRIEF DESCRIPTION OF THE DRAWINGS

In order to more clearly illustrate the embodiments of the present disclosure or the technical solutions in the prior art, the drawings that are required to be used in the embodiments or the descriptions of the prior art will be briefly described below, and it is obvious that the drawings in the following descriptions are only embodiments of the present disclosure, and that other drawings can be obtained according to the provided drawings without inventive effort for a person skilled in the art.

FIG. 1 is an SEM image of the initial inner surface of a PVC pipeline before coating modification of example 1, as observed under a scanning electron microscope.

FIG. 2 is an SEM image of the surface (A) and the surface partial enlargement (C), the pipeline section (B) and the partial enlargement (D) of the zwitterionic polymer coating in the PVC pipeline prepared in example 1, as observed under a scanning electron microscope.

FIG. 3 PU@PSB, PDMS@PSB and PET@PSB are SEM images of the surface structures of the coatings prepared in examples 16, 18 and 19, respectively, with 200 μm scale.

FIG. 4 is an illustration of the anti-platelet activation properties of samples before and after modification of the zwitterionic polymer coating of example 1.

FIG. 5 is an illustration of the anti-platelet adhesion properties of samples before and after modification of the zwitterionic polymer coating of example 1.

FIG. 6 is a graph showing the change in bacterial adhesion on the surface of samples before and after modification of the zwitterionic polymer coating of example 1.

FIG. 7 shows the change in friction coefficient and relative protein adhesion of the zwitterionic polymer coating prepared in example 1, when the rolling by peristaltic pump lasted for different times.

FIG. 8 is SEM images of the surface morphology of the coating prepared in example 1 when the rolling by peristaltic pump lasted different times.

FIG. 9 shows the change in friction coefficient of the surface of the coating prepared in example 1 under the action of long-time shearing force.

FIG. 10 shows the thrombosis of PVC pipeline before and after modification of the zwitterionic polymer coating in example 1 after 12 hours of extracorporeal circulation of Guangxi Bama miniature pig. (a) an extracorporeal circulation schematic diagram of Guangxi Bama miniature pig, (b) a photograph of thrombus adhesion of a polymer coated modified PVC pipeline (left) and an uncoated modified PVC pipeline (right) after extracorporeal circulation for 12 hours; (c) the amount of thrombus in both pipelines.

FIG. 11 (a) is a physical picture of the extracorporeal circulation of Guangxi Bama miniature pig; (b) In-line whole Blood adhesion SEM images after 12 hours of extracorporeal circulation in Guangxi Bama miniature pig: uncoated modified PVC, PU pipe, coated modified pipeline (PVC@PSB) in example 1 and coated modified pipeline (PU@PSB) in example 17.

FIG. 12 shows the changes in the activated partial thromboplastin time (APTT), prothrombin time (PT) and thrombin time (TT) of blood, after the PVC pipeline modified by the zwitterionic polymer coating of example 1 was used for various times of the extracorporeal circulation of Guangxi Bama miniature pig.

FIG. 13 shows the changes in fibrinogen content, red blood cell count, white blood cell count, and platelet count in the blood, after the PVC pipeline modified by the zwitterionic polymer coating of example 1 was used for various times of the extracorporeal circulation of Guangxi Bama miniature pig.

FIG. 14 is a graph showing the mechanical stability of the coating having a crosslinked structure prepared in example 1, the coating having no crosslinked structure of example 22, and the coating having a low crosslinked structure prepared in example 23, which are observed by a confocal microscope, and the scale was 200 μm.

FIG. 15 is an SEM image of the microstructure of the coating surface of example 31.

FIG. 16 is an illustration of the anti-platelet adhesion and anti-platelet activation properties of the coating of example 31.

FIG. 17 is an illustration of the anticoagulant properties of the coated modified polyurethane pipeline in extracorporeal blood circulation prepared in example 31.

FIG. 18 (a) is physical picture of the extracorporeal circulation of Guangxi Bama miniature pig; (b) after 12 hours of extracorporeal circulation of Guangxi Bama miniature pig, whole Blood adhesion SEM images of the inner surface of uncoated modified PVC, coated modified PVC pipeline prepared in example 35.

FIG. 19 is SEM images of the coating surfaces under different tests comparing the mechanical stability of the coatings of example 1 and example 35.

FIG. 20 is a graph showing the thickness and friction coefficient of the coating at various times during which polymerization was initiated by the method in example 1.

### DETAILED DESCRIPTION OF THE EMBODIMENTS

The following description of the technical solutions in the embodiments of the present disclosure will be clear and complete, and it is obvious that the described embodiments are only some embodiments of the present disclosure, but not all embodiments. All other embodiments, which can be made by those skilled in the art based on the embodiments of the disclosure without making any inventive effort, are intended to be within the scope of the disclosure.

The technical scheme disclosed by the disclosure is further described below with reference to specific embodiments.

### Example 1

The preparation of the zwitterionic polymer coating on the inner wall of the PVC pipeline includes the following steps:

Firstly, activating the surface of a substrate: the medical grade PVC pipeline (with the inner diameter of 12 mm and the wall thickness of 1 mm) was flushed with isopropanol and deionized water and then completely dried by nitrogen flow; one end of the pipeline was sealed, then the other end was infused with ethanol solution of 20 wt % benzophenone and then soaked for 3 minutes at 25° C., after which the redundant benzophenone and the ethanol solution were recycled, and the pipeline was cleaned by ethanol and dried by nitrogen.

Secondly, preparing a precursor solution: zwitterionic monomers SBMA, a crosslinking agents MBA and photo initiators Irgacure 2959 were dissolved into deionized water to prepare a zwitterionic polymer precursor solution; in the precursor solution, the SBMA content was 20 wt %, the mass percentage of the crosslinking agent MBA to the zwitterionic being 10%, and the mass percentage of the photo initiator Irgacure 2959 to the zwitterionic was 10%.

Thirdly, initiating graft crosslinking polymerization: after sealing one end of the PVC pipeline with activated surface, the zwitterionic polymer precursor solution was injected into the inner cavity of the pipeline at the other end, and the pipeline was uniformly irradiated by ultraviolet light with wavelength of 365 nm (850 mW/cm²) at 20-25° C. for 50 minutes, and then the adsorbate on the inner surface of the tube was repeatedly washed away with a large amount of deionized water. The sample was dried at room temperature with nitrogen, sterilized with H₂O₂ low-temperature plasma, and packed.

### Examples 2 to 18

According to the three steps of example 1, namely, activating the surface of the substrate, preparing a precursor solution and initiating graft crosslinking polymerization, the zwitterionic polymer coating of the disclosure could be prepared on different substrates by changing the substrate materials and the types and the amounts of monomers, crosslinking agents and initiators and adjusting the process parameters. The technology of the disclosure adopted photoinitiated polymerization, and could be carried out at room temperature.

The coating preparation conditions and parameters of examples 2-18 are set forth in table 1, and it is noted that the activated substrate may be rinsed with water or a volatile solvent (ethanol, isopropanol, acetone, etc.).

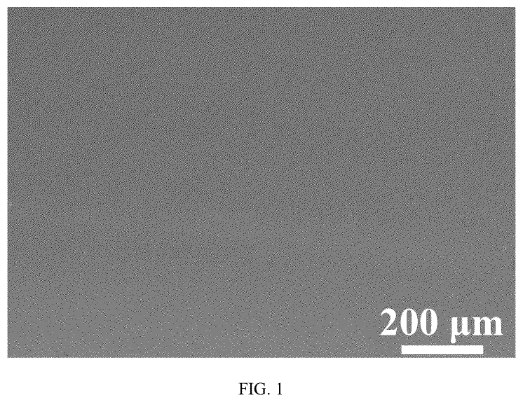

The structure and properties of the prepared PVC, PU, PET and PDMS surface coatings are shown in FIGS. 1-13 and table 2, and the corresponding test methods are described later in this specification.

TABLE 1
Preparation process parameters and conditions for the coatings of examples 2-15

| | Activating substrate | | | | | Precursor Solution | | | Graft Crosslinking | |
|---|---|---|---|---|---|---|---|---|---|---|
| | | Surface | | | | | Crosslinking | | Polymerization | |
| | | Initiator/ | | Temperature | Time | Monomer/ | agent^{a}/ | Initiator^{b}/ | Temperature | Time |
| Example | Substrate | wt % | Solvent | (° C.) | (min) | wt % | wt % | wt % | (° C.) | (min) |
| 2 | PVC | BP/20 | Ethanol | 20 | 5 | SBMA/20 | MBA/4 | Irg/10 | 25 | 50 |
| 3 | PVC | BP/20 | Ethanol | 20 | 5 | SBMA/10 | MBA/3 | Irg/1 | 25 | 50 |
| 4 | PVC | BP/20 | Ethanol | 25 | 3 | SBMA/15 | MBA/10 | Irg/15 | 20 | 40 |
| 5 | PVC | BP/30 | Ethanol | 25 | 3 | SBMA/40 | MBA/9 | Irg/10 | 25 | 50 |
| 6 | PVC | BP/30 | Ethanol | 25 | 3 | SBMA/60 | MBA/12 | Irg/20 | 28 | 60 |
| 7 | PVC | BP/30 | Ethanol | 25 | 3 | SBMA/20 | MSBA/10 | Irg/0.5 | 30 | 100 |
| 8 | PVC | BP/15 | Isopropanol | 30 | 4 | SBMA/20 | MBA/12 | Irg/15 | 30 | 60 |
| 9 | PVC | MBP/10 | Acetonitrile | 40 | 4 | CBMA/20 | MSBA/8 | Irg/10 | 25 | 45 |
| 10 | PVC | MBP/30 | Acetonitrile | 25 | 3 | CBMA/40 | MSBA/12 | Irg/10 | 25 | 40 |
| 11 | PVC | BP/20 | Acetone | 20 | 4 | MPC/20 | MBA/10 | Irg/10 | 28 | 60 |
| 12 | PVC | IPTH/5 | Acetone | 25 | 10 | SBMA/20 | EBA/10 | Krt/8 | 25 | 45 |
| 13 | PVC | BP/20 | Ethanol | 25 | 3 | SBMA/20 | CBBA/10 | Krt/15 | 25 | 40 |
| 14 | PVC | BP/20 | Acetone | 20 | 4 | MPC/20 | MBA/3 | Irg/10 | 30 | 60 |
| 15 | PU-t1 | BP/20 | Ethanol | 25 | 3 | SBMA/20 | MBA/5 | Irg/10 | 25 | 50 |
| 16 | PU-t2 | MBP/10 | Acetonitrile | 30 | 5 | MPC/20 | CBBA/8 | Irg/10 | 25 | 40 |
| 17 | PU-t3 | BP/10 | Ethanol | 30 | 5 | SBMA/20 | MBA/10 | Irg/10 | 25 | 40 |
| 18 | PDMS | IPTH/5 | Acetone | 25 | 10 | CBMA/30 | EBA/12 | Krt/8 | 25 | 45 |
| 19 | PET | BP/20 | Ethanol | 30 | 5 | SBMA/20 | MSBA/10 | Irg/10 | 25 | 50 |
| 20 | PA | BPO/15 | DMSO | 30 | 10 | SBMA/20 | MBA/10 | APS/8 | 80 | 60 |
| 21 | PDMS | AIBN/10 | THF | 30 | 10 | MPC/20 | CBBA/8 | PPS/10 | 80 | 60 |

BP: benzophenone;
MBP: 4-methylbenzophenone;
IPTH: 2-isopropylthioxanthone;
BPO: benzoyl peroxide;
APS: ammonium persulfate;
AIBN: azobisisobutyronitrile;
THF: tetrahydrofuran;
DMSO: dimethyl sulfoxide;
Irg: Irgacure 2959;
krt: α-ketoglutaric acid;
APS: ammonium persulfate;
PPS: potassium persulfate;
PU-t1, PU-t2 and PU-t3 were respectively small-caliber polyurethane pipes, and the sizes were respectively (diameter/wall thickness): 10 mm/0.75 mm, 6 cm/0.75 mm, 3 cm/0.5 mm;
PET pipeline: 10 mm/0.7 mm; PDMS pipeline: 5 mm/0.5 mm
^{a}the crosslinking agent in the precursor solution accounted for the mass percent of the zwitterionic monomer;
^{b}the initiator in the precursor solution accounted for the mass percent of the zwitterionic monomer.

### Example 19

The three steps of example 1 were followed, except that a polyethylene terephthalate (PET) plate (2 cm*2 cm*0.5 cm) was immersed in a hydrophobic initiator to activate the surface, and the activated PET plate was immersed in a zwitterionic polymer precursor solution to photoinitiated graft crosslinking polymerization to obtain a surface coating. The structural properties of the coating were measured as shown in table 1.

### Example 20

The three steps of example 1 were followed, except that a polyamide (PA) membrane was immersed in a dimethyl sulfoxide (DMSO) solution of the hydrophobic surface initiator benzoyl peroxide to activate the surface, rinsed with distilled water, and air dried; the activated PA membrane was immersed into a zwitterionic polymer precursor solution to thermally initiated graft crosslinking polymerization for 1 h at 80° C. by adopting ammonium sulfate as a water-soluble initiator to obtain the surface coating. The structural properties of the coatings were measured as shown in tables 1 and 2.

### Example 21

Zwitterionic coating modifications were performed on a silicone rubber (PDMS) membranes as in example 20, except that the surface of the PDMS membrane was activated with a solution of azobisisobutyronitrile (AIBN) in tetrahydrofuran (THF), and potassium persulfate (PPS) was used as a water-soluble initiator to obtain a surface coating, as shown in tables 1 and 2.

The changes in protein adhesion amount, platelet adhesion amount, friction coefficient, and activated partial thromboplastin time (APTT), prothrombin time (PT), and thrombin time (TT) of the various kinds of polymer coatings of the above examples were measured, as shown in Table 2.

CF₀/CF₁=initial friction coefficient of coating/friction coefficient after 10 days of PBS solution shearing.

The results in Table 2 show that the surface friction coefficient, surface modulus and protein adhesion were all greatly reduced after the coating modification was performed compared with the surface of the original substrate. The thickness of the prepared zwitterionic polymer coating was 25-100 μm; the friction coefficient in an aqueous medium was less than 0.005; the surface Young's modulus was 10-60 kPa; the protein adhesion amount was very low; the platelet adhesion was zero, and the anticoagulation performance was high. The friction coefficient after 10 days of PBS solution shearing (CF₁) essentially unchanged, and only in examples 2, 3 and 14, when the dosage of crosslinking agent was low, CF₀/CF₁ was less than 1 but higher than 0.5, indicating that the coating had high stability.

TABLE 2
Zwitterionic polymer coating properties prepared from different monomers

| | | | | | Adₚₗₐ/ | | | | | | |
|---|---|---|---|---|---|---|---|---|---|---|---|
| | | | | | Adₚₗₐ₋₁₀ | | | | | | Hemolysis |
| | T | | E | Adₚᵣₒ | (PCs./ | APTT | PT | TT | CF₀/ | Adₚᵣₒ₋₁₀ | Rate |
| Example | (μm) | CF₀ | (kPa) | (μg/cm²) | mm²) | (s) | (s) | (s) | CF₁ | (μg/cm²) | (%) |
| 1 | 83 ± | 0.0020 | 25.3 ± | 0.20 ± | 0/0 | 35.64 | 17.3 | 23.4 | 1.003 | 0.20 ± | 1.1 |
| | 9 | | 1.32 | 0.05 | | | | | | 0.04 | |
| 2 | 40 ± | 0.0020 | 10.0 ± | 0.30 ± | 0/0 | 33.6 | 16.4 | 16.2 | 0.87 | 0.35 ± | 0.7 |
| | 6 | | 1.02 | 0.05 | | | | | | 0.07 | |
| 3 | 25 ± | 0.0045 | 13.4 ± | 0.20 ± | 0/3 ± 1 | 32.1 | 16.2 | 16.7 | 0.540 | 0.73 ± | 0.7 |
| | 3 | | 1.25 | 0.03 | | | | | | 0.05 | |
| 4 | 55 ± | 0.0041 | 23.0 ± | 0.18 ± | 0/0 | 33.0 | 17.1 | 18.2 | 1.002 | 0.19 ± | 1.5 |
| | 9 | | 1.55 | 0.04 | | | | | | 0.06 | |
| 5 | 92 ± | 0.0022 | 33.5 ± | 0.20 ± | 0/0 | 35.6 | 17.4 | 23.2 | 1.001 | 0.21 ± | 1.0 |
| | 10 | | 1.98 | 0.04 | | | | | | 0.05 | |
| 6 | 100 ± | 0.0019 | 59.8 ± | 0.15 ± | 0/0 | 30.6 | 16.4 | 16.2 | 1.000 | 0.15 ± | 2.0 |
| | 12 | | 2.06 | 0.05 | | | | | | 0.06 | |
| 7 | 62 ± | 0.0042 | 19.3 ± | 0.24 ± | 0/0 | 33.6 | 16.4 | 16.2 | 0.955 | 0.23 ± | 1.0 |
| | 5 | | 1.96 | 0.06 | | | | | | 0.03 | |
| 8 | 72 ± | 0.0043 | 39.3 ± | 0.30 ± | 0/0 | 32.64 | 16.3 | 21.6 | 1.000 | 0.27 ± | 2.0 |
| | 5 | | 1.32 | 0.05 | | | | | | 0.07 | |
| 9 | 70 ± | 0.0025 | 27.9 ± | 0.23 ± | 0/0 | 35.2 | 15.2 | 18.5 | 1.000 | 0.23 ± | 1.0 |
| | 5 | | 2.63 | 0.02 | | | | | | 0.05 | |
| 10 | 80 ± | 0.002 | 23.9 ± | 0.27 ± | 0/0 | 33.50 | 16.2 | 18.6 | 0.992 | 0.25 ± | 1.9 |
| | 6 | | 1.26 | 0.03 | | | | | | 0.07 | |
| 11 | 72 ± | 0.002 | 27.2 ± | 0.15 ± | 0/0 | 38.24 | 19.3 | 26.3 | 1.005 | 0.19 ± | 0.7 |
| | 3 | | 1.35 | 0.02 | | | | | | 0.02 | |
| 12 | 82 ± | 0.0041 | 28.9 ± | 0.19 ± | 0/0 | 33.2 | 16.1 | 17.7 | 0.982 | 0.20 ± | 1.0 |
| | 10 | | 2.02 | 0.04 | | | | | | 0.04 | |
| 13 | 85 ± | 0.0015 | 20.0 ± | 0.17 ± | 0/0 | 32.4 | 15.9 | 18.3 | 1.000 | 0.18 ± | 1.4 |
| | 4 | | 1.12 | 0.05 | | | | | | 0.04 | |
| 14 | 32 ± | 0.0018 | 10.0 ± | 0.32 ± | 0/1 ± 1 | 33.2 | 16.6 | 15.3 | 0.687 | 0.30 ± | 0.6 |
| | 5 | | 0.60 | 0.05 | | | | | | 0.05 | |
| 15 | 47 ± | 0.0022 | 28.4 ± | 0.21 ± | 0/0 | 34.0 | 18.0 | 23.4 | 0.968 | 0.24 ± | 0.9 |
| | 3 | | 1.35 | 0.03 | | | | | | 0.02 | |
| 16 | 56 ± | 0.0020 | 26.4 ± | 0.19 ± | 0/0 | 35.14 | 18.3 | 24.1 | 1.001 | 0.19 ± | 0.7 |
| | 2 | | 1.31 | 0.02 | | | | | | 0.05 | |
| 17 | 54 ± | 0.0022 | 23.4 ± | 0.20 ± | 0/0 | 35.64 | 17.3 | 23.4 | 1.003 | 0.21 ± | 1.1 |
| | 3 | | 1.15 | 0.03 | | | | | | 0.03 | |
| 18 | 80 ± | 0.0035 | 32.5 ± | 0.16 ± | 0/0 | 35.34 | 17.6 | 22.9 | 1.000 | 0.17 ± | 1.4 |
| | 9 | | 4.32 | 0.03 | | | | | | 0.05 | |
| 19 | 74 ± | 0.0028 | 33.6 ± | 0.19 ± | 0/0 | 36.02 | 18.3 | 23.9 | 1.000 | 0.20 ± | 1.0 |
| | 7 | | 1.93 | 0.02 | | | | | | 0.04 | |
| 20 | 44 ± | 0.0042 | 23.4 ± | 0.30 ± | 0/0 | 30.34 | 16.3 | 20.8 | 0.913 | 0.41 ± | — |
| | 3 | | 1.15 | 0.03 | | | | | | 0.09 | |
| 21 | 36 ± | 0.0047 | 21.4 ± | 0.37 ± | 0/0 | 31.60 | 16.0 | 21.1 | 0.909 | 0.47 ± | — |
| | 4 | | 1.05 | 0.06 | | | | | | 0.05 | |
| PVC | — | 1.024 | 410.4 | 85.8 | — | 13.4 | 9.1 | 14.66 | — | — | 3.56 |
| PU | — | 1.10 | 150.0 | 75.3 | — | — | — | — | — | — | 3.23 |
| PET | — | 1.65 | 541.7 | 65.9 | — | — | — | — | — | — | 3.67 |
| PDMS | — | 0.89 | 124.3 | 65.2 | — | — | — | — | — | — | 2.31 |
| PA | — | 0.91 | 620.5 | 96.3 | — | — | — | — | — | — | 2.87 |

T: thickness of the coating;
E: Young's modulus of coating surface;
CF₀: initial friction coefficient of coating;
CF₁: friction coefficient of coating after 10 days of PBS solution shearing;
Adₚᵣₒ: initial protein adhesion amount;
Adₚᵣₒ₋₁₀: protein adhesion amount after 10 days of PBS solution shearing under peristaltic pump;
Adₚₗₐ: platelet adhesion amount;
Adₚₗₐ₋₁₀: platelet adhesion amount after 10 days of PBS solution shearing under peristaltic pump.
TT: thrombin time;
APTT: activated partial thromboplastin time;
PT: prothrombin time.
Control group: APTT, PT and TT for platelet poor plasma were 31 s, 14. s and 18 s, respectively.

As can be seen from the data in table 2, the coating still showed low protein adhesion and zero platelet adhesion after 10 days of PBS solution shearing. In particular, when the concentration of the zwitterionic monomer in the precursor was preferably 15-40 wt %, the crosslinking agent accounting for 5-10 wt % of the mass of the zwitterionic monomer, and the water-soluble initiator accounted for 1-15 wt % of the mass of the zwitterionic monomer, the graft crosslinking polymerization reaction was better controlled, making the polymerization time shorter and the coating performance better.

APTT, PT, TT are important parameters for evaluating the anticoagulation performance of a material, and the higher the values, the better the anticoagulation performance. The data in table 2 shows a significant increase in APTT, PT, TT after modification with the zwitterionic polymer coating compared to the original substrate surface (PVC), which further demonstrates the excellent anticoagulant properties of the coating.

The results in Table 2 also show that the prepared zwitterionic polymer coating had very low hemolysis rate, which was below 2%, better blood compatibility and met the requirements of the hemolysis rate of medical instruments.

In order to further prove the structure and performance of the coating, the structure of the coating was characterized by adopting transmission electron microscope, scanning electron microscope, confocal microscope and the like, and the application performances of serial intracorporal and extracorporeal blood compatibility, anticoagulation, mechanical stability and the like were also characterized. The specific method is described later in the specification, and the results are shown in FIG. 1-FIG. 12.

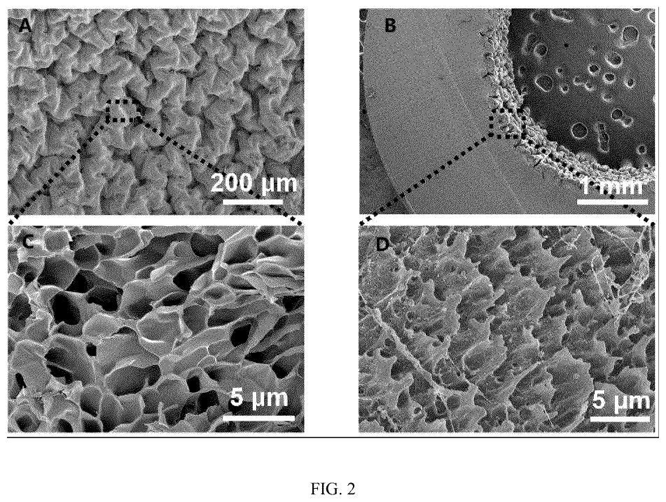

As can be seen from FIG. 1, the inner surface of the PVC pipeline before the coating modification was of a smooth structure. FIG. 2 (A, B) shows that after the zwitterionic polymer coating modification, the surface exhibited a uniform micron-sized crosslinked groove structure and “paving-stone” like protrusions, with the coating thickness being 50-100 μm; the SEM image was further enlarged to show that the surface was in a nano-micron pore structure; the zwitterionic hydrogel had super-hydrophilicity, absorbing water to form a hydration layer, and the nano-scale pore wall swung along the direction of water flow, so that the hydrogel had the anti-adhesion property of super-lubrication on the surface.

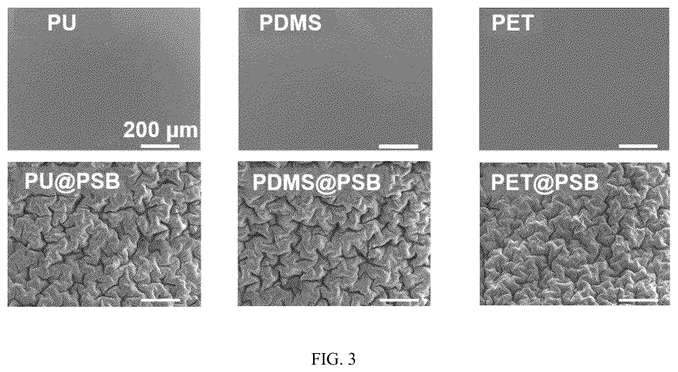

FIG. 3 shows that coatings with microscale crosslinked groove structures could be formed on the surfaces of PU, PET, PDMS polymer substrates using different surface initiators, zwitterionic monomers, crosslinking agents and water-soluble initiators in accordance with the method of the present disclosure.

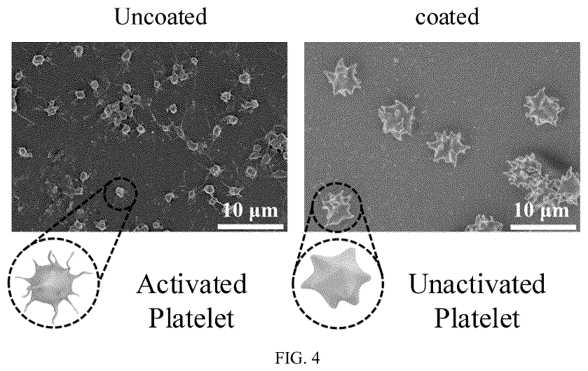

Visible in FIG. 4: on the unmodified PVC surface, platelets were activated and adhered to the surface, specifically as follows: platelets extended pseudopodia and began to deform as a whole, adhering to each other and aggregating into a mass. After modification with the zwitterionic polymer coating, the surface platelets were in an unactivated resting state.

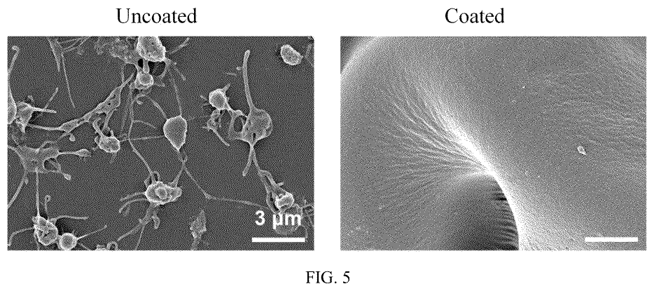

Visible in FIG. 5: after being modified by the zwitterionic polymer coating, the surface had no platelet adhesion, so that the prepared zwitterionic polymer coating had the performance of zero platelet activation and zero platelet adhesion.

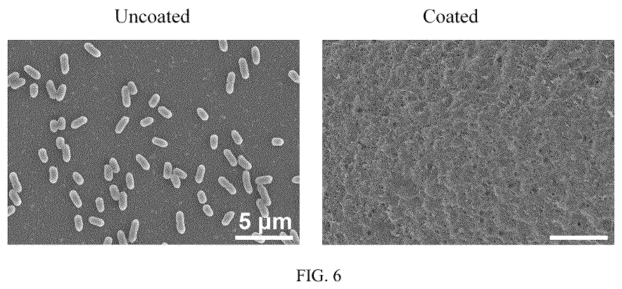

As can be seen from FIG. 6: after being modified by the zwitterionic polymer coating, the surface was free of adhesion of Escherichia coli, which shows that the prepared zwitterionic polymer coating had excellent antibacterial adhesion resistance.

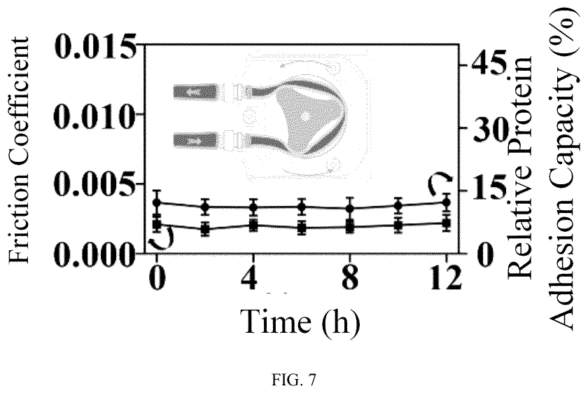

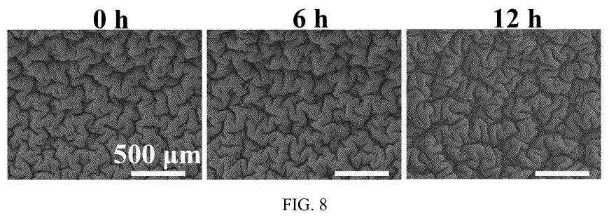

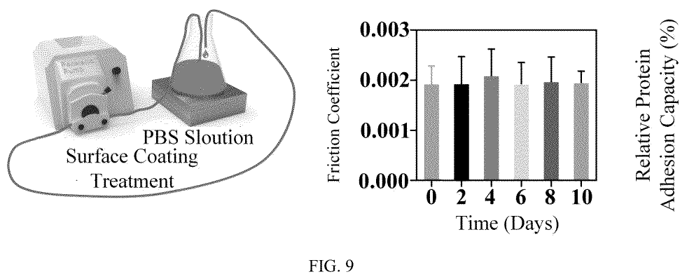

As can be seen from FIG. 7: after being subjected to the peristaltic pump for 12 hours, the super-lubricating property (friction coefficient) and the protein adhesion resistance of the coating were unchanged. As can be seen from FIG. 8: after being subjected to the peristaltic pump for 12 hours, the surface morphology of the coating was unchanged. As can be seen from FIG. 9: under the action of PBS solution shearing force, the friction coefficient of the surface of the polymer coating was hardly changed after 10 days, and the polymer coating still had super-lubrication performance.

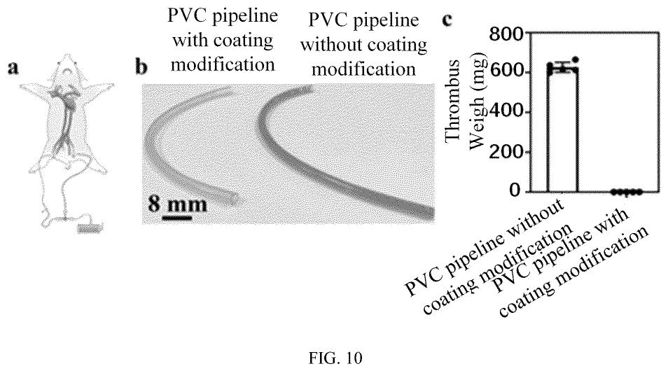

FIG. 10 shows thrombosis of PVC pipeline before and after modification of the zwitterionic polymer coating in example 1 after 12 hours of extracorporeal circulation of Guangxi Bama miniature pig. It can be seen that: after the modification by the zwitterionic polymer coating, the pipeline was free from any thrombus, so that the prepared zwitterionic polymer coating could effectively inhibit the formation of thrombosis in the extracorporeal circulation process.

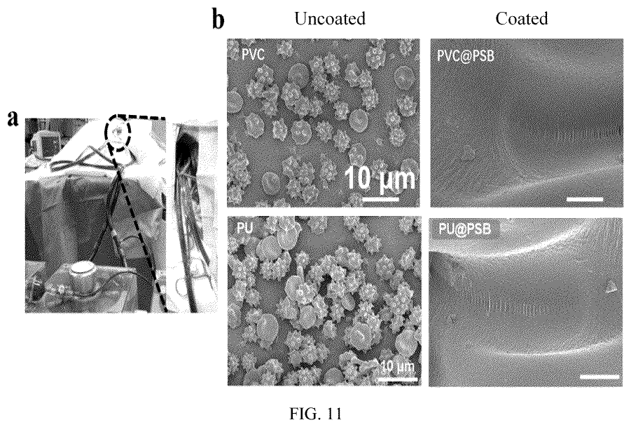

As can be seen from FIG. 11: after the extracorporeal circulation of Guangxi Bama miniature pig for 12 hours, PVC and PU modified by the zwitterionic polymer coating had no platelet adhesion inner the pipelines, however, obvious thrombus formed by blood cell adhesion and aggregation could be seen in the pipelines without coatings, so that the prepared zwitterionic polymer coatings could effectively inhibit activation and adhesion of platelets in the extracorporeal circulation process.

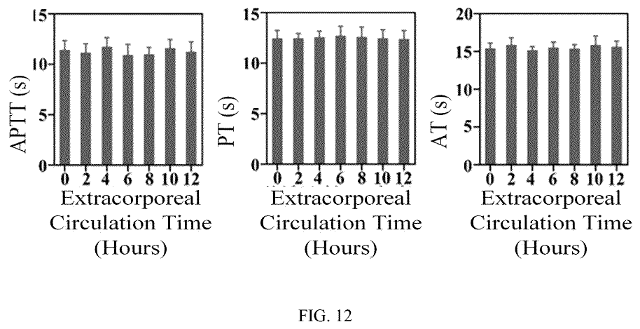

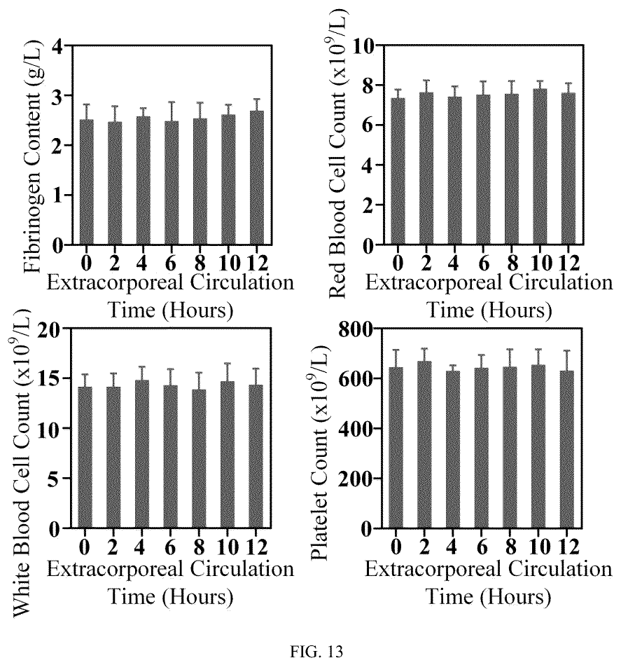

As can be seen from FIG. 12: within 12 hours of the extracorporeal circulation of the Guangxi Bama miniature pig, APTT, PT, TT of the blood did not change significantly, demonstrating that the prepared zwitterionic polymer coating exhibited a “blood undisturbed” property during the extracorporeal circulation. As can be seen from FIG. 13: within 12 hours of the extracorporeal circulation of the Guangxi Bama miniature pig, the fibrinogen content, the red blood cell number, the white blood cell number and the platelet number of the blood did not change obviously, which further proved the prepared zwitterionic polymer coating had the “blood undisturbed” property in the extracorporeal circulation process.

To further illustrate the performance advantages of the zwitterionic polymer coatings of the present disclosure, a comparison was made with the zwitterionic coating techniques reported previously, such as comparative example 1 and comparative example 2.

### Comparative Example 1

By the layer-by-layer assembly and surface grafting method reported previously (J Mater Chem, B, 2019, 7 (39): 6024-6034; CN 110643277A), zwitterionic polymer coatings with different thicknesses were built on PVC pipeline surfaces. The process is as follows: the PVC pipeline was washed with ethanol and deionized water respectively in an ultrasonic condition for three times, and left to dry at room temperature. Then, the PVC pipeline was soaked in a 2 mg/mL dopamine solution, reacting at 37° C. for 24 hours, and then the PVC pipeline was taken out and rinsed with deionized water, and dried with N₂ to obtain a PDA-coated pipeline PVC@PDA. The PVC@PDA pipeline was immersed into 30 mg/ml of 3-aminopropyltriethoxysilane (APTES) hydrolysate, soaking for 12 hours at 37° C., rinsing with deionized water and drying to obtain an organosilicon coated PVC pipeline, PVC@PDA/Si. Finally, the PVC@PDA/Si pipeline was immersed into a 10 mg/mL solution of a methacryloyl ethyl sulfobetaine polymer with epoxy groups (PSBG_{42/4}) for 24 hours at 60° C., and rinsed and dried to obtain a PVC pipeline with a surface graft zwitterionic polymer coating: PVC@PDA/Si/PSB-1, as shown in Table 3.

### Comparative Example 2

According to the literature method (Macromol. Biosci. 2018, 18, 1700359), a zwitterionic polymer molecular brush coating was grafted onto the inner surface of PVC pipeline. The inner surface of the PVC pipeline was washed twice with ethanol and deionized water, and then oxygen plasma treatment was performed for 5 min to activate the surface; the pipeline was immersed into 1 mg/mL an 11-(trichlorosilyl) undecyl-2-bromo-2-methylpropanoate toluene solution at 25° C. for 1 h, then washed with toluene, acetone, ethanol and deionized water, and dried with nitrogen; then, the PVC pipeline was filled with a pre-formulated zwitterionic polymer precursor solution (dissolving 15.5 mmol SBMA and 0.7 mol NaBr into a mixture of 7.27 mL dimethyl sulfoxide and 15.0 ml water, and then adding 391 μL of CuBr2 mother liquor (3.9 μmol CuBr₂, 23.4 μmol 3-(2-dimethylaminoethyl) amine, and 10.0 mL DMSO)), and at room temperature, 365 nm ultraviolet light was uniformly irradiated for 15 minutes to initiate a graft polymerization reaction; then the reaction solution was removed, the PVC pipeline was washed with DMSO, acetone, ethanol and a large amount of deionized water, and dried with nitrogen to obtain a surface graft-polymerized zwitterionic polymer molecular brush coating PVC-g-PSB, as shown in Table 3.

TABLE 3
Performance control of zwitterionic polymer coatings with different thicknesses prepared by different methods

| | T | E | | | CF₀/ | Adₚᵣₒ | Adₚᵣₒ₋₁₀ | Adₚₗₐ | Adₚₗₐ₋₁₀ |
|---|---|---|---|---|---|---|---|---|---|
| Coating | (μm) | (kPa) | CF₀ | CF₁ | CF₁ | (μg/cm²) | (ug/cm²) | (PCs./mm²) | (PCs./mm²) |
| Example 1 | 83 ± 9 | 25.3 ± 1.32 | 0.002 | 0.002 | 1.00 | 0.22 ± 0.05 | 0.20 ± 0.04 | 0 | 0 |
| Control | 0.325 ± 0.017 | 127.5 ± 1.58 | 0.28 | 0.68 | 0.41 | 9.2 ± 0.9 | 43.6 ± 0.9 | 162 ± 15 | 1062 ± 35 |
| Group 1 | | | | | | | | | |
| Control | 0.066 ± 0.022 | 189.6 ± 1.66 | 0.22 | 1.88 | 0.12 | 8.0 ± 0.4 | 153.2 ± 0.6 | 153 ± 22 | 1553 ± 42 |
| Group 2 | | | | | | | | | |

T: thickness of the coating;
E: Young's modulus of coating surface;
CF₀: initial friction coefficient of coating;
CF₁: friction coefficient of coating after 10 days of PBS solution shearing;
Adₚᵣₒ: initial protein adhesion amount;
Adₚᵣₒ₋₁₀: protein adhesion amount after 10 days of PBS solution shearing;
Adₚₗₐ: platelet adhesion amount;
Adₚₗₐ₋₁₀: platelet adhesion amount after 10 days of PBS solution shearing.

The data in Table 3 shows that the coating obtained by the conventional preparation method (examples 2 and 3) of the molecular brush coating of the surface graft polymer was relatively thin, ranging from about 300 to 500 nm, and the surface elastic modulus of the coating was relatively high, above 100 kPa, under the influence of the PVC substrate (the surface elastic modulus was 400 kPa); platelet and protein adhesion amounts were much higher than the zwitterionic polymer coatings of example 1; especially, with poor stability of the coating, the friction coefficient of the PBS solution was increased sharply after shearing for 10 days, and further the adhesion of surface proteins was greatly improved, which indicated that the coating was dropped. In contrast, the zwitterionic polymer coating prepared by the surface graft crosslinking method of example 1 exhibited a suitable thickness (about 83 μm), a surface elastic modulus with soft elastic characteristics (about 25 kPa), an extremely low friction coefficient of 0.002, showing zero platelet adhesion and extremely low protein adhesion properties, and the coating stability was good. Thus, the zwitterionic polymer coating with surface graft crosslinking of the present disclosure represented a significant advancement.

Further, the technology of the present disclosure was also used to prepare the coating using non-zwitterionic water-soluble monomers, such as comparative examples 3-6, as shown in Table 4.

### Comparative Examples 3 to 6

The process of example 1 was followed, using the same crosslinking agent and initiator as in example 1, except that the kind of water-soluble monomer used was changed (table 4), to obtain a surface coating as shown in table 4.

TABLE 4
Zwitterionic polymer coating properties prepared from different monomers

| | | | | Adₚₗₐ | | | | | | |
|---|---|---|---|---|---|---|---|---|---|---|
| | Monomer | T | Adₚᵣₒ | (PCs./ | | E | CF₀/ | APTT | PT | TT |
| Coating | Type | (μm) | (μg/cm²) | mm²) | CF₀ | (kPa) | CF₁ | (s) | (s) | (s) |
| Example 1 | SBMA | 83 ± 9 | 0.20 ± 0.05 | 0 | 0.002 | 25.3 ± 1.32 | 1.003 | 35.64 | 17.3 | 23.4 |
| Control | AA | 69 ± 9 | 4.63 ± 0.55 | 7550 | 0.032 | 140.0 ± 6.51 | 0.99 | 19.57 | 8.3 | 8.2 |
| Group 3 | | | | | | | | | | |
| Control | AAm | 81 ± 7 | 3.02 ± 0.60 | 6520 | 0.021 | 166.2 ± 5.00 | 1.062 | 14.85 | 8.0 | 9.2 |
| Group 4 | | | | | | | | | | |
| Control | VP | 76 ± 10 | 5.08 ± 0.43 | 9570 | 0.027 | 145.7 ± 4.55 | 0.985 | 16.55 | 9.4 | 8.2 |
| Group 5 | | | | | | | | | | |
| Control | HEMA | 90 ± 5 | 4.63 ± 0.30 | 8500 | 0.019 | 136.2 ± 9.85 | 0.997 | 19.08 | 6.5 | 5.9 |
| Group 6 | | | | | | | | | | |

AA: acrylic acid;
AAm: acrylamide;
VP: 1-vinyl-2-pyrrolidone;
HEMA: hydroxyethyl methacrylate

The data in table 4 shows that the polymer coating formed from zwitterionic monomer (SBMA, CBMA, MPC) had soft elastic characteristics, a surface elastic modulus of less than 100 kPa, a friction coefficient of less than 0.005, and showed very excellent protein adhesion resistance, platelet adhesion resistance (zero platelet adhesion), anticoagulant properties, which benefited from the higher hydrophilicity of the zwitterionic polymer and extremely low interactions with cells, proteins and the like, as compared to polymer coatings formed from several non-zwitterionic monomers, such as acrylic acid, acrylamide, 1-vinyl-2-pyrrolidone, hydroxyethyl methacrylate.

The effect of the amount of the crosslinking agent on the coating properties was also studied. The procedure of example 1 was followed, except that the amount of the crosslinking agent was changed to obtain surface coatings, and the changes in the protein adhesion amount, platelet adhesion amount, friction coefficient and surface Young's modulus of the different coatings were measured as shown in tables 5 and 6.

TABLE 5
Analysis of the performance of zwitterionic polymer coatings prepared with
different dosage of crosslinking agent

| | | | | | Adₚₗₐ | | | | | |
|---|---|---|---|---|---|---|---|---|---|---|
| | MBA | T | E | Adₚᵣₒ | (PCs./ | | CF₀/ | APTT | PT | TT |
| Example | Dosage^{a} | (μm) | (kPa) | (μg/cm²) | mm²) | CF₀ | CF₁ | (s) | (s) | (s) |
| 22 | 0 | 10 ± 2 | 1.6 ± 0.25 | 0.22 ± 0.06 | 0 | 0.0015 | 0.028 | 32.3 | 15.4 | 15.3 |
| 23 | 0.5% | 25 ± 5 | 2.1 ± 0.36 | 0.40 ± 0.08 | 0 | 0.0017 | 0.055 | 32.5 | 16.3 | 15.5 |
| 24 | 3% | 72 ± 4 | 10.0 ± 0.60 | 0.32 ± 0.05 | 0 | 0.0018 | 0.54 | 33.2 | 16.6 | 16.1 |
| 25 | 5% | 78 ± 6 | 10.0 ± 1.02 | 0.30 ± 0.05 | 0 | 0.0020 | 0.87 | 33.6 | 16.4 | 16.2 |
| 26 | 6% | 80 ± 9 | 12.4 ± 1.00 | 0.28 ± 0.09 | 0 | 0.0020 | 0.95 | 33.4 | 16.2 | 17.5 |
| 27 | 9% | 83 ± 5 | 19.2 ± 1.45 | 0.23 ± 0.1 | 0 | 0.0022 | 1.001 | 35.3 | 16.3 | 19.2 |
| 28 | 12% | 89 ± 7 | 29.6 ± 1.65 | 0.25 ± 0.03 | 0 | 0.0023 | 1.000 | 35.5 | 17.4 | 18.2 |
| 29 | 13% | 93 ± 9 | 35.5 ± 1.32 | 0.52 ± 0.07 | 96 ± 15 | 0.0037 | 1.001 | 23.8 | 12.5 | 15.4 |

TABLE 6
Comparison of mechanical stability of crosslinked versus uncrosslinked coatings

| | | | Friction Coefficient After | | | Adₚᵣₒ (μg/cm²) After | | |
|---|---|---|---|---|---|---|---|---|
| | | | Treatment | | | Treatment | | |
| | | | | Rapid | | | Rapid | |
| | | | | Water | | | Water | |
| | Initial | | Peristaltic | Flow | | Peristaltic | Flow | |
| | Friction | Adₚᵣₒ | Pump | Scouring | Repeated | Pump | Scouring | Repeated |
| Example | Coefficient | (μg/cm²) | for 12 h | for 24 h | Bending | for 12 h | for 24 h | Bending |
| 1 | 0.002 | 0.20 | 0.002 | 0.002 | 0.002 | 0.21 | 0.20 | 0.20 |
| 22 | 0.0015 | 0.22 | 0.40 | 0.60 | 0.50 | 80 | 100 | 60 |
| 23 | 0.0017 | 0.40 | 0.017 | 0.012 | 0.010 | 32 | 40 | 35 |
| 24 | 0.0018 | 0.32 | 0.0037 | 0.002 | 0.0025 | 0.70 | 0.45 | 0.56 |

The data in table 5 illustrates that the amount of crosslinking agent MBA had a greater effect on the properties of the coatings. Without the crosslinking agent (example 22), the coating was thinner (10 μm), and although it also had zero platelet adhesion properties, the coating stability was poor, and the friction coefficient increased dramatically (approximately 2.1 for PVC surface friction coefficient) after 10 days of PBS solution shearing. Namely, the ratio CF₀/CF₁ was very low, which indicated that the coating was peeled, and thus the platelet adhesion and the protein adhesion were dramatically increased. However, the hydrophilicity decreased due to excessive crosslinking agent, resulting in increased platelet adhesion, as in example 29.

The data in table 5 further demonstrates that at crosslinking agent below 3%, the coating stability was poor, and after 10 days of PBS solution shearing, the platelet and protein adhesion levels were increased, and the coagulation parameters APTT, PT and TT were all significantly reduced. When the crosslinking agent was 5%-12%, the coating stability was better, and the anti-adhesion and anticoagulation functions of the coating were not affected basically after 10 days of the PBS solution shearing, so that the method was suitable for long-time extracorporeal blood circulation application. However, too high crosslinking, e.g., 13%, may result in too dense a coating, with reduced performance, and too high a crosslinking agent may be detrimental to control of the polymerization reaction.

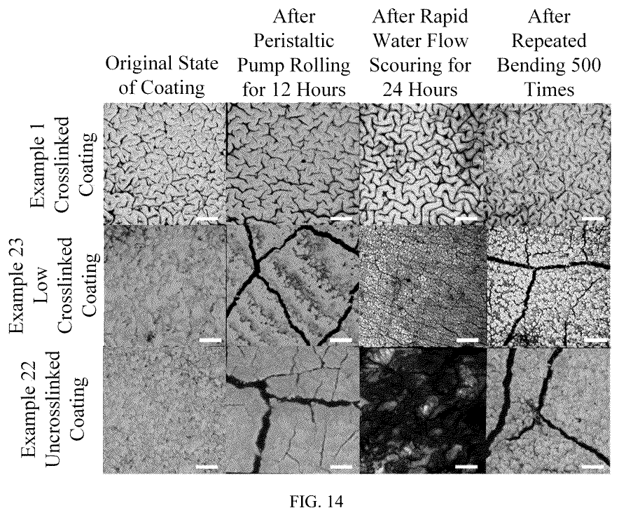

FIG. 14 and table 6 further compare the mechanical stability of the coatings of the crosslinked structure with that of the uncrosslinked structure. In the application process, the coating needs to withstand the rolling friction of a peristaltic pump, the great blood scouring action and the repeated bending action, so that better mechanical stability and firmness are needed. The data in FIG. 14 and table 6 illustrate that, while also having a surface micro-nanostructure, lower friction coefficient and better protein adhesion resistance in the initial state, after rolling of the peristaltic pump and bending, the uncrosslinked zwitterionic polymer coating prepared in example 22 obviously cracked, along with the friction coefficient increasing and the protein adhesion resistance decreasing. The coating had peeled off under rapid water washout (FIG. 14), also resulting in an increase in the friction coefficient and a decrease in the protein adhesion resistance (table 6). At 3% crosslinking agent, the mechanical stability was greatly improved (table 6). The coating with the crosslinked structure prepared in the example 1 could well resist rolling, water scouring and bending, the coating structure staying intact, and the friction coefficient and the protein adhesion resistance were not affected, so that the zwitterionic polymer coating with surface graft crosslinking prepared in the disclosure has good mechanical stability and can meet the application requirements of long-time blood circulation.

### Examples 30 to 35d

According to the method of example 1, the operation was the same as that of example 1. The difference was that the composition of the precursor solution was changed, a physical crosslinking agent N-acryloylglycine amide (NAGA) was added, and the inner surface of polyurethane pipeline (PU-t2) or PVC pipeline were modified by the coatings. The specific compositions and process parameters are shown in table 7.

TABLE 7
Preparation process parameters and conditions for the coatings of examples 30-40

| | | Precursor Solution | | | | | |
|---|---|---|---|---|---|---|---|
| | | | | Physical | | | |
| | | | | Crosslinking | | Graft Crosslinking | |
| | | | Chemical | Agent | | Polymerization | |
| | | Monomer/ | Crosslinking | NAGA*/ | Initiator*/ | Temperature | Time |
| Example | Pipeline | wt % | Agent*/wt % | wt % | wt % | (° C.) | (min) |
| 30 | PU-t2 | SBMA/20 | MBA/10 | 10% | Irg/10 | 25 | 50 |
| 31 | PU-t2 | SBMA/20 | MBA/10 | 20% | Irg/10 | 25 | 50 |
| 32 | PU-t2 | SBMA/30 | MBA/10 | 40% | Irg/5 | 20 | 50 |
| 33 | PU-t2 | SBMA/40 | MBA/7 | 30% | Irg/15 | 25 | 45 |
| 34 | PU-t2 | SBMA/60 | MBA/5 | 40% | Irg/20 | 28 | 50 |
| 35 | PVC | SBMA/20 | MSBA/10 | 15% | Irg/10 | 25 | 50 |
| 36 | PVC | CBMA/40 | MBA/12 | 5% | Irg/12 | 30 | 50 |
| 37 | PU-t2 | CBMA/15 | MSBA/8 | 20% | Irg/1 | 25 | 75 |
| 38 | PU-t2 | MPC/20 | MSBA/10 | 30% | Irg/10 | 25 | 40 |
| 39 | PVC | MPC/40 | CBBA/10 | 20% | Irg/10 | 28 | 60 |
| 40 | PVC | SBMA/20 | MBA/10 | 45% | Irg/10 | 25 | 50 |

*the amount of the crosslinking agent and the initiator in the precursor solution was the mass percentage of the zwitterionic monomer;

TABLE 8
Zwitterionic Polymer coating properties prepared with different levels of physical crosslinking agent

| | | | | | Adₚₗₐ/ | | | | | | |
|---|---|---|---|---|---|---|---|---|---|---|---|
| | | | | | Adₚₗₐ₋₂₀ ^{a} | | | | | | Hemolysis |
| | T | | E | Adₚᵣₒ | (PCs./ | APTT | PT | TT | CF₀/ | Adₚᵣₒ₋₂₀ ^{a} | Rate |
| Example | (μm) | CF₀ | (kPa) | (μg/cm²) | mm²) | (s) | (s) | (s) | CF₂ ^{b} | (μg/cm²) | (%) |
| 1 | 83 ± 9 | 0.0020 | 25.3 ± | 0.20 ± | 0/21 ± 3 | 35.6 | 17.3 | 23.4 | 0.067 | 43 ± 4 | 1.1 |
| | | | 1.32 | 0.05 | | | | | | | |
| 30 | 80 ± 8 | 0.0023 | 29.5 ± | 0.22 ± | 0/2 ± 2 | 35.1 | 17.0 | 23.3 | 0.853 | 0.57 ± | 1.0 |
| | | | 1.08 | 0.03 | | | | | | 0.05 | |
| 31 | 89 ± 6 | 0.0023 | 33.3 ± | 0.24 ± | 0/0 | 34.2 | 17.1 | 22.9 | 1.003 | 0.28 ± | 0.9 |
| | | | 1.24 | 0.04 | | | | | | 0.03 | |
| 32 | 96 ± 5 | 0.0024 | 36.9 ± | 0.37 ± | 0/0 | 35.7 | 16.9 | 22.8 | 1.000 | 0.36 ± | 1.3 |
| | | | 2.32 | 0.08 | | | | | | 0.05 | |
| 33 | 89 ± 5 | 0.0027 | 39.4 ± | 0.30 ± | 0/0 | 34.6 | 18.0 | 22.7 | 0.997 | 0.33 ± | 1.0 |
| | | | 1.07 | 0.04 | | | | | | 0.05 | |
| 34 | 105 ± 8 | 0.0030 | 42.3 ± | 0.27 ± | 0/0 | 34.7 | 17.9 | 23.5 | 0.882 | 0.47 ± | 0.8 |
| | | | 1.96 | 0.08 | | | | | | 0.08 | |
| 35 | 82 ± 4 | 0.0032 | 30.8 ± | 0.36 ± | 0/0 | 35.0 | 17.5 | 23.1 | 0.961 | 0.35 ± | 1.0 |
| | | | 1.25 | 0.08 | | | | | | 0.09 | |
| 36 | 96 ± 4 | 0.0043 | 49.5 ± | 0.35 ± | 0/20 ± 5 | 33.7 | 16.4 | 22.3 | 0.246 | 0.68 ± | 0.8 |
| | | | 2.65 | 0.07 | | | | | | 0.05 | |
| 37 | 46 ± 5 | 0.0035 | 53.6 ± | 0.30 ± | 0/0 | 31.9 | 16.0 | 21.9 | 0.887 | 0.31 ± | 1.5 |
| | | | 1.58 | 0.06 | | | | | | 0.04 | |
| 38 | 92 ± 6 | 0.0038 | 60.2 ± | 0.32 ± | 0/0 | 33.5 | 17.6 | 21.3 | 1.001 | 0.30 ± | 1.2 |
| | | | 1.03 | 0.08 | | | | | | 0.06 | |
| 39 | 96 ± 6 | 0.0027 | 30.5 ± | 0.25 ± | 0/0 | 33.9 | 17.0 | 21.6 | 1.002 | 0.24 ± | 0.9 |
| | | | 1.40 | 0.03 | | | | | | 0.03 | |
| 40 | 87 ± 7 | 0.0150 | 85.5 ± | 0.85 ± | 0/12 ± 4 | 26.4 | 15.0 | 20.4 | 0.421 | 1.85 ± | 1.7 |
| | | | 1.33 | 0.03 | | | | | | 0.03 | |

^{a} Adₚₗₐ₋₂₀ and Adₚᵣₒ₋₂₀ are the platelet adhesion and protein adhesion amount of the coatings after 20 days of PBS solution shearing under peristaltic pump, respectively;
^{b} CF₂: friction coefficient after 20 days of PBS solution shearing under peristaltic pump

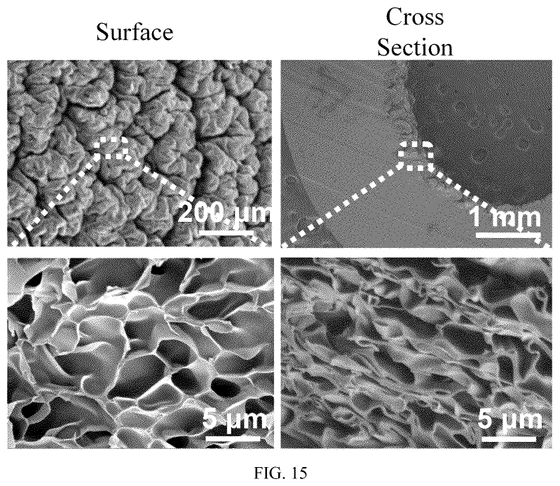

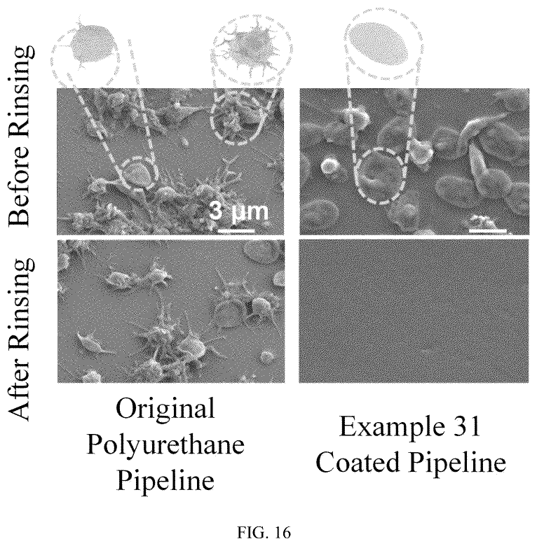

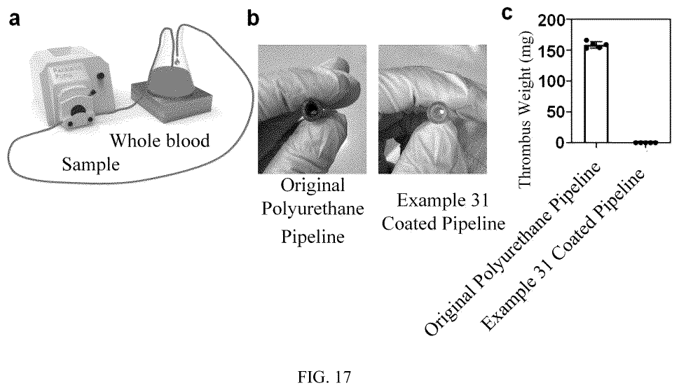

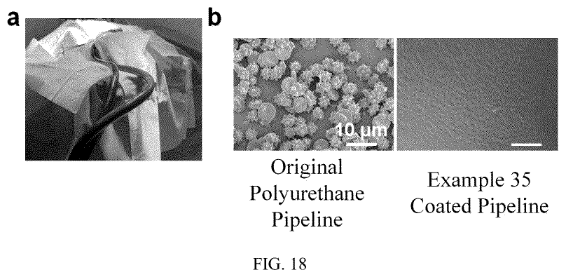

Table 8 further compares the properties of coatings with different physical crosslink densities. The addition of a proper amount of physical crosslinking agent NAGA did not affect the excellent anti-bioadhesion of the coating, and could also obviously enhance the stability and durability of the coating. The data in table 8 shows that when the physical crosslinking agent NAGA accounting for 5 to 40 wt % of the mass of the zwitterionic monomer was added into the precursor solution according to the technical scheme of the disclosure, a zwitterionic polymer coating with the thickness of 25 to 100 μm, the friction coefficient in an aqueous medium of less than 0.005 and the surface Young's modulus of 10 to 60 kPa could be formed on the surfaces of PVC and polyurethane substrates, and the coating had a micro-nano surface groove structure (as shown in FIG. 15). The data in table 8 also shows that the coating had very low protein adhesion and zero platelet adhesion at levels of physical crosslinking agent NAGA less than 40 wt % of the mass of zwitterionic monomer, and FIG. 16 further shows the zero platelet adhesion and platelet activation resistance of the coating surface with NAGA. The three coagulation parameters (APTT, PT, TT) in table 8 demonstrate that the introducing of the physically crosslinked coating still had better anticoagulation performance, and FIG. 17 is the anticoagulation result of the coating-modified polyurethane pipeline with extracorporeal blood circulation prepared in example 31, further demonstrating that the coating with chemical crosslinking and physical crosslinking did not cause thrombus. While, FIG. 18 demonstrates that the inner surface of the coating-modified PVC pipeline prepared in example 35 did not cause blood cell adhesion after 12 hours of extracorporeal circulation of Guangxi Bama miniature pig.

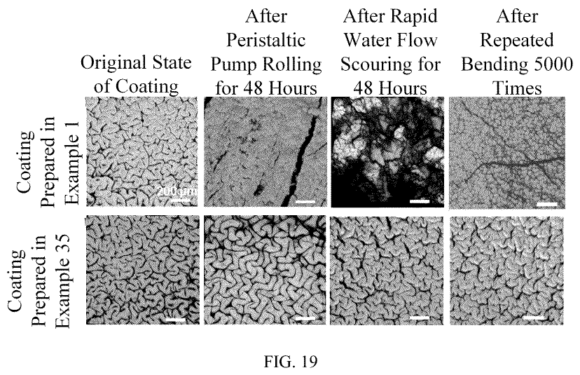

The physical crosslinking agent NAGA is introduced into the coating, and the most obvious advantage is to improve the mechanical stability of the coating, so that the coating is suitable for blood circulation application for a longer time. FIG. 19 compares the mechanical stability of the coating of example 1 (having only a chemically crosslinked structure) with the coating of example 35 (having both chemically crosslinked and physically crosslinked structure), and shows that the coating of example 1 with only a chemically crosslinked structure was cracked and peeled off while the coating of example 35 still maintained a relatively intact surface structure after increasing the rolling time of peristaltic pump to 48 h, rapid water scouring for 48 h and bending for 5000 times, which demonstrates that the reversible hydrogen bonding physical crosslinking network formed by NAGA unit could better resist mechanical wear and damage, and endowed the coated pipeline with longer service performance. In Table 8, the ratio of the surface friction coefficient (CF₀/CF₂), protein adhesion amount Adₚᵣₒ₋₂₀ and platelet adhesion amount Adₚₗₐ₋₂₀ after 20 days of PBS solution shearing under the peristaltic pump further explained that only in example 1 of chemical crosslinking, the CF₀/CF₂ of the coating was far less than 1 due to the coating falling off after 20 days of shearing, and the protein and platelet adhesion increased significantly, while the coating added NAGA maintained good resistance to protein adhesion and platelet adhesion, especially when the NAGA dosage accounted for 10 wt %-40 wt % of zwitterionic monomer, CF₀/CF₂ gradually approaching 1, maintaining zero platelet adhesion performance.

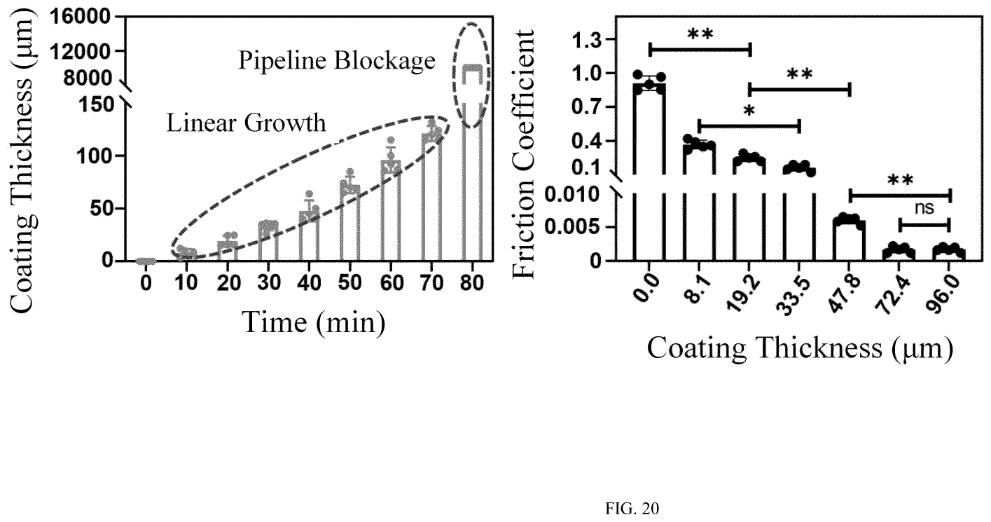

The preferred embodiments of the present disclosure have been described in detail above, but the present disclosure is not limited to the specific details of the above embodiments, and various simple modifications can be made to the technical solution of the present disclosure within the scope of the technical concept of the present disclosure, and all the simple modifications belong to the protection scope of the present disclosure. The thickness of the coating can be regulated and controlled according to the requirement by the initiation time, the dosage of the crosslinking agent and the monomer, and is related to the surface area and the size of the pipeline and also related to the intensity of the ultraviolet light used. FIG. 20 shows the coating thickness and friction coefficient obtained by ultraviolet initiated polymerization for different times according to the method of example 1. With the extension of the initiation time, the coating thickness increased linearly, and the friction coefficient gradually decreased; after 40 minutes, the coating friction coefficient dropped below 0.005, and then stabilized; too long a polymerization initiation time (80 min) may cause a line blockage due to an increase in the crosslinked polymer in the solution.

Moreover, any combination of the various embodiments of the disclosure can be made without violating the spirit of the disclosure, which should also be considered as disclosed herein.

And, the characterization methods of the zwitterionic polymer coating prepared by the disclosure have the following specific contents:

### 1) Analysis of the Surface Morphology and Thickness of the Coating Under a Scanning Electron Microscope:

The surface morphology and thickness of the coating were observed by means of a field emission scanning electron microscope (SEM) (HITACHI S-4800, Hitachi). Prior to SEM characterization, all samples to be tested were surface gold-plated under argon for 60 seconds to enhance the conductivity of the samples. And the surface morphology and the section thickness of the sample were observed under the condition that the accelerating voltage was 3 kV and the working distance was 10-15 mm.

### 2) Protein Adhesion Test:

The test method used for the protein adhesion test is the BCA protein kit method. The principle is that under alkaline conditions, when BCA binds to protein, the protein will reduce Cu²⁺ to Cu⁺, and one Cu⁺ can chelate two BCA molecules. Therefore, the working reagent forms a purple complex from the original apple green. It has a high absorbance at 562 nm and is proportional to the protein concentration. The protein used in this experiment was bovine serum albumin (BSA) which is commonly used. According to the instructions of the BCA protein kit, a series of standard protein solutions of 0, 2.5, 5, 10, 20, 40, 200 μg/mL were prepared. The absorbance of the standard protein solution at the wavelength of 562 nm was measured, and finally a protein BSA standard curve was drawn by taking the absorbance as an abscissa and the protein concentration as an ordinate.

### 3) Surface Young's Modulus Test:

The surface Young's modulus of the modified zwitterionic polymer coating on the substrate was measured by a desktop PIUMA nanoindenter, and a spherical indentation probe with a radius of 48.5 mm was used to probe the sample immersed in PBS, with 5×5 point scanning, point-to-point spacing of 20 μm, and detection area of 100×100 μm.

### 4) Surface Friction Coefficient Test:

The surface friction coefficient of the zwitterionic polymer coating was measured by a CSM-friction wear testing machine. A sample was put into constant-temperature deionized water at 25° C. in advance, and a test probe (glass ball with the diameter of 3 mm) slid on the surface to be measured at the sliding speed of 30 mm/min, with a sliding distance of 20 mm. The friction coefficient was calculated from the friction divided by the corresponding normal load (800 UN).

### 5) Biocompatibility Testing:

### (1) Platelet Activation Test:

Firstly, rabbit blood was collected, and separated for 15 min at a centrifugal speed of 1500 r/min by a high-speed centrifuge, and supernatant was sucked as platelet-rich-plasma (PRP) for later use. The prepared samples were respectively placed into 24 pore plates, 60 μL of PRP respectively sucked by using a pipettor and uniformly dripped onto the surfaces of the samples, and the samples were placed into a constant-temperature water bath box at 37° C. for shaking incubation for 1 h. The samples were immersed in 2.5 wt % glutaraldehyde solution, fixed at 4° C. overnight, and taken out from the glutaraldehyde solution. 50%, 75%, 90% and 100% absolute ethanol solutions were prepared respectively, and the air-dried samples were soaked in the gradient absolute ethanol solutions respectively for dehydration for 15 min each time. The morphology of platelets on the surface was observed by scanning electron microscope.

### (2) Platelet Adhesion Test:

PRP preparation and platelet adhesion on the material surface were the same as above, but the difference was that after adhesion, the samples needed to be washed five times with PBS for 1 min each time. After cleaning, the samples were taken out, immersed in 2.5 wt % glutaraldehyde solution, fixed at 4° C. overnight, and the samples were taken out from the glutaraldehyde solution and air-dried. 50%, 75%, 90% and 100% absolute ethanol solutions were prepared respectively, and the air-dried samples were soaked in the gradient absolute ethanol solutions respectively for dehydration for 15 min each time. The number of surface-adhered platelets was observed using a scanning electron microscope.

### (3) Bacterial Adhesion Test:

The samples (1×1 cm²) were rinsed three times with PBS, sterilized for 30 min under UV irradiation, placed in a 24-well plate and covered with 1 mL of bacterial suspension (108 CFU/mL). The same were cultured in an incubator at 37° C. for 4 hours. The substrate was then washed three times with PBS to remove any unattached bacteria. Bacteria were fixed overnight at 4° C. with 2.5 wt % glutaraldehyde, which was blotted after fixing, then the samples were rinsed three times gently with PBS and dehydrated continuously with 50%, 75%, 95% and 100% ethanol for 10 minutes. The samples were dried and observed under a scanning electron microscope to observe three different locations on each sample and count the average number of adherent bacteria.

### (4) Hemolysis Rate Test:

The extent of destruction of blood cells (mainly erythrocytes) under the zwitterionic polymer coating was evaluated by the hemolysis rate test. A sample to be tested was filled into a test tube, and 10 mL of 0.9% NaCl solution was added; distilled water was used as the positive control, and 0.9% NaCl solution was used as the negative control. Fresh ACD anticoagulated rabbit blood (rabbit blood: 3.8% sodium citrate=4:1) was adopted; all test tubes were placed into a 37° C. water bath for preheating for 30 min; 0.2 mL of fresh anticoagulated rabbit blood (rabbit blood: physiological saline=4:5) was added and diluted respectively, then all test tubes were kept in the 37° C. water bath for 1 h and centrifuged for 5 min (2500 r/min); the supernatant was taken, and the absorbance value of each tube was measured at 545 nm of the spectrophotometer. Hemolysis=(sample absorbance-positive control absorbance)/(negative control absorbance-positive control absorbance). If the hemolysis rate was less than 5%, it means that the zwitterionic polymer coating met the hemolysis rate requirement of the medical material.

### (5) Prothrombin Time (PT):

The effect of the zwitterionic polymer coating on the coagulation time due to the activation of the prothrombin factor was evaluated using the prothrombin time assay. Platelet-rich-plasma (PRP) was adding into a test tube by Quick method, then 0.1 mL of rabbit brain extract was added, and the test tube was placed in a water bath at 37° C. for 2 min; 0.1 mL of 0.025 mol/L CaCl₂) solution with the temperature being preheated to 37° C. was added; simultaneously timing was started; immediately the test tube was shaken for several times and immersed in a water bath; the test tube was removed from the water bath for 5-8s, and tilted continuously until coagulation occurred, which was the coagulation time. Each test tube and control tube were averaged over 3 times.

### (6) Activated Partial Thromboplastin Time (APTT):

The extent of activation of the endogenous coagulation factor by the zwitterionic polymer coating was evaluated by an activated partial thromboplastin time test, thereby evaluating its effect on coagulation time. The sample was cut into squares of 0.5 cm×0.5 cm, placed in a 1.5 mL centrifuge tube, added with 0.5 mL of PBS, incubated at 37° C. for 1 h at rest, and then the PBS was subsequently aspirated. 20 mL of rabbit blood was collected by a vacuum blood collection tube, and 3.2% sodium citrate was added for anticoagulation (v:v=1:9); the anticoagulated peripheral blood was centrifuged with a centrifuge at 4000 rmp for 10 min, then the upper platelet-poor-plasma (PPP) of the centrifuged blood was collected; 400 μL of PPP was taken and added into a centrifuge tube containing the sample; another 400 μL of plasma was added into a 1.5 mL blank centrifuge tube as an experimental control, and a static incubation was performed for 30 min in a 37° C. constant-temperature water bath. Subsequently, the incubated PPP was pipetted into a new 1.5 mL centrifuge tube; 0.1 mL of plasma and 0.1 mL of an action reagent (pre-warmed at 37° C. and prepared from cephalin plus 1×10-4M ellagic acid, buffer, stabilizer and preservative) were automatically pipetted using a fully automated coagulation analyzer (cs5100, SYSMEX, Japan) and mixed thoroughly, incubated at 37° C. for 3 min, and then 0.1 mL of 25 mM CaCl₂) solution was pipetted into the instrument, then the solution was mixed thoroughly and timing was started while the instrument detected coagulation formation and the APTT was calculated automatically.

### (7) Thrombin Time (TT):

The effects of the zwitterionic polymer coating on coagulation, anticoagulation and function of the fibrinolytic system in blood were evaluated through the thrombin time test. The sample was cut into squares of 0.5 cm×0.5 cm, placed in a 1.5 mL centrifuge tube, added with 0.5 mL of PBS, incubated at 37° C. for 1 h at rest, and then the PBS was subsequently aspirated. 20 mL of rabbit blood was collected by a vacuum blood collection tube, and 3.2% sodium citrate was added for anticoagulation (v:v=1:9); the anticoagulated peripheral blood was centrifuged with a centrifuge at 4000 rmp for 10 min, then the upper platelet-poor-plasma (PPP) of the centrifuged blood was collected; 400 μL of PPP was taken and added into a centrifuge tube containing the sample; another 400 μL of plasma was added into a 1.5 mL blank centrifuge tube as an experimental control, and a static incubation was performed for 30 min in a 37° C. constant-temperature water bath. Subsequently, the incubated PPP was pipetted into a new 1.5 mL centrifuge tube; 0.1 mL of plasma was automatically pipetted into a test cup using a fully automated coagulation analyzer (cs5100, SYSMEX, Japan), incubated at 37° C. for Imin, then the instrument was allowed to aspirate 0.2 mL of thrombin (pre-warmed at 37° C. and prepared from 1.5 IU/mL of bovine thrombin, bovine albumin), then the solution was mixed thoroughly and timing was started while the instrument detected coagulation time and the TT was calculated.

### (8) Extracorporeal Circulation Experiment of Guangxi Bama Miniature Pig

An extracorporeal circulation experiment was performed using a Guangxi Bama miniature pig arteriovenous shunt model, and 0.2 mg of scopolamine was injected intramuscularly before anesthesia. Intramuscular injection of haloperidol 5 mg and ketamine 20 mg/kg induced anesthesia were performed. The ear margin was intravenous injected with propofol, fentanyl and seccolin to maintain anesthesia, and the trachea cannula was inserted after successful anesthesia. After anesthesia, the right femoral artery and the left femoral vein were exposed, then blood was introduced into the body through an extracorporeal circulation pipeline; an arteriovenous shunt model was built, after circulation was carried out for 12 hours, the extracorporeal circulation pipeline was taken down, and the incision was sutured. The inner surface of the pipeline was observed for whole Blood adhesion under SEM.

### 6) Coating Stability Test:

### (1) Test of Rolling Resistance of Coating

The peristaltic pump was used to roll the pipeline modified zwitterionic polymer coating at 50 rpm (FIG. 7); the rolled locations were sampled at different time points; the friction coefficient and protein adhesion resistance of the sample surface were measured, and the surface morphology changes before and after coating rolling were observed under SEM.

### (2) Coating Scouring Resistance Test

\textcircled{1} Simulating a blood flow shear state: the mechanical stability of the polymer coating was verified through peristaltic pump extracorporeal circulation experiments; under the action of a peristaltic pump, PBS solution was used for scouring the inner surface of a pipeline at the flow rate of 3 mL/s; materials were obtained at different time points, and the friction coefficient change of the surface of a sample was measured to determine the scouring resistance of the coating.
\textcircled{2} Rapid water scouring experiment

Samples of the surface modified by coating (2 cm×2 cm) were placed horizontally. The water flow scoured the sample vertically at a rate of 1.5 m/s. The distance between the nozzle and the sample was 30 cm. After a certain period of scouring, the surface morphology was measured after drying at room temperature.

### (3) Bending Resistance Test:

The mechanical stability of the hydrogel coating was evaluated by repeated fold-unfold cyclic deformation tests. The specific experimental steps were as follows: the PVC pipeline (length 20 cm, inner diameter 4 mm) with the inner surface modified by coating was subjected to multiple fold-unfold cyclic tests at the same position, wherein the folding angle was 180 degrees, and the folding cycle was 2s each time. After bending for a certain number of times, the surface morphology was measured.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

What is claimed is:

1. A zwitterionic polymer coating with surface graft crosslinking, wherein the polymer coating is a coating formed on a surface of a substrate by simultaneously initiating a graft crosslinking polymerization of a zwitterionic monomer and a water-soluble crosslinking agent in a surface of a polymer substrate and an aqueous solution which have been activated in advance by a surface initiator; the aqueous solution consists of a zwitterionic monomer, a water-soluble crosslinking agent and a water-soluble initiator
wherein a thickness of the polymer coating is 25-100 μm; and
a friction coefficient of the polymer coating in an aqueous medium is less than 0.005, and a surface Young's modulus is 10-60 kPa; and
the zwitterionic monomer is at least one of methacryloyl ethyl sulfobetaine, 2-methacryloyl oxyethyl phosphorylcholine and carboxylic acid betaine methacrylate; and
the water-soluble crosslinking agent comprises a chemical crosslinking agent accounting for 3 wt % to 12 wt % of the mass of the zwitterionic monomer, and a physical crosslinking agent accounting for 0 wt % to 40 wt % of the mass of the zwitterionic monomer;
the chemical crosslinking agent is at least one of N, N-methylenebisacrylamide, N, N-bis (acryloyl) cystamine, ethylene glycol dimethacrylate, and carboxylate betaine dimethacrylate, and the physical crosslinking is selected from N-acryloylglycine amide;
the surface initiator is a hydrophobic photo initiator or thermal initiator selected from benzophenone, 4-methyl benzophenone, isopropyl thioxanthone, benzoyl peroxide or azodiisobutyronitrile; the water-soluble initiator is a photo initiator or a thermal initiator selected from Irgacure-2959, α-ketoglutaric acid, ammonium persulfate or potassium persulfate.

2. A zwitterionic polymer coating with surface graft crosslinking of claim 1, wherein the concentration of the zwitterionic monomer is 10 wt % to 60 wt %; the water-soluble initiator accounts for 0.5 wt % to 20 wt % of the mass of the zwitterionic monomer.

3. A zwitterionic polymer coating with surface graft crosslinking of claim 1, wherein a material of the polymer substrate is selected from polyvinyl chloride, polyurethane, polyester, polyamide or rubber.

4. The zwitterionic polymer coating with surface graft crosslinking of claim 2, wherein a concentration of the zwitterionic monomer in the aqueous solution is 15 wt % to 40 wt %; the chemical crosslinking agent accounts for 5 wt % to 10 wt % of the mass of the zwitterionic monomer; the physical crosslinking agent accounts for 10 wt % to 40 wt % of the mass of the zwitterionic monomer, and the water-soluble initiator accounts for 1 wt % to 15 wt % of the mass of the zwitterionic monomer.

5. A preparation method for a zwitterionic polymer coating with surface graft crosslinking of claim 1, wherein a surface initiator is first swelled into a surface of a substrate for activation and then a graft crosslinking polymerization of a zwitterionic monomer and a water-soluble crosslinking agent is jointly initiated in the surface activated by the surface initiator and an aqueous solution to obtain a surface coating; the preparation method comprises the following specific steps:
(1) soaking a substrate in a solution of a surface initiator to diffuse the surface initiator into a surface layer of the substrate, activating the surface of the substrate, flushing the surface of the substrate with deionized water or a solvent, and drying;
(2) immersing the surface of the substrate activated by the surface initiator in the step (1) into a zwitterionic polymer precursor solution, performing a graft crosslinking polymerization by photo or thermal initiation, and then flushing an adsorbate on the surface by deionized water to form a zwitterionic polymer coating with a graft crosslinking structure on the surface of the substrate;
wherein the precursor solution is an aqueous solution composed of a zwitterionic monomer, a water-soluble crosslinking agent and a water-soluble initiator, the water-soluble crosslinking agent comprises a chemical crosslinking agent and a physical crosslinking agent; the concentration of the zwitterionic monomer is 10 wt %-60 wt %; the chemical crosslinking agent accounts for 3 wt %-12 wt % of the mass of the zwitterionic monomer; the physical crosslinking agent accounts for 0 wt %-40 wt % of the mass of the zwitterionic monomer, and the water-soluble initiator accounts for 0.5 wt %-20 wt % of the mass of the zwitterionic monomer.

6. The preparation method for a zwitterionic polymer coating with surface graft crosslinking of claim 5, wherein the step of performing a graft crosslinking polymerization by photo initiation is as follows:
(1) immersing the modified surface of the substrate in a surface photo initiator solution, performing activation treatment on the surface of the substrate, then flushing the surface of the substrate with deionized water or a solvent, and drying;
(2) immersing the surface of the substrate activated by the surface initiator into a zwitterionic polymer precursor solution, uniformly irradiating the surface of the substrate and the precursor solution by ultraviolet light to initiate a graft crosslinking polymerization, and then flushing the surface of the substrate by deionized water to form a zwitterionic polymer coating with a graft crosslinking structure on the surface of the substrate;
wherein the surface photo initiator is selected from benzophenone, 4-methyl benzophenone or isopropyl thioxanthone; the water-soluble initiator is selected from Irgacure-2959, α-ketoglutaric acid.

7. An application of the zwitterionic polymer coating with surface graft crosslinking of claim 1 for modifying inner or outer surfaces of materials, articles and devices, to impart anti-bioadhesion and anticoagulant functions to the surfaces.

8. The application of claim 7, further comprising: the application of the zwitterionic polymer coating with surface graft crosslinking in the surface modification of various medical devices.

9. The application of claim 8, wherein the medical devices contains pipelines, artificial blood vessels.
